(19) United States
(12) Patent Application Publication
(10) Pub. No.: US 2023/0150960 A1
(43) Pub. Date: May 18, 2023

(54) COMPOSITIONS AND METHODS OF USING CYANINE DYES TO TARGET CANCER AND MITOCHONDRIA

(71) Applicant: Thomas Jefferson University, Philadelphia, PA (US)

(72) Inventors: Avinoam Nevler, Narberth, PA (US); Christopher William Schultz, Riverton, NJ (US); Jonathan Robert Brody, Philadelphia, PA (US)

(21) Appl. No.: 17/796,570

(22) PCT Filed: Jan. 29, 2021
(86) PCT No.: PCT/US2021/015768

§ 371 Date: Jul. 29, 2022

Related U.S. Application Data
(60) Provisional application No. 62/967,768, filed on Jan. 30, 2020.

(51) Int. Cl. C07D 277/64; A61K 45/06; C07D 263/56; G16B 5/20; A61K 31/428; A61K 31/423; G16B 25/10

(52) U.S. Cl. CPC C07D 277/64; A61K 31/423; A61K 31/428; A61K 45/06; C07D 263/56; G16B 5/20; G16B 25/10

(57) ABSTRACT

Described herein are compounds of Formula I and methods of using said compounds for treating, ameliorating, and/or preventing cancer. The compounds are cyanine dyes, and have increased effects in hypoglycemic environments.

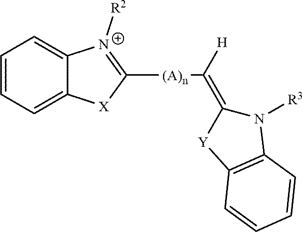

### CROSS-REFERENCE TO RELATED APPLICATION

This application claims priority to U.S. Provisional Pat. Application Serial No. 62/967,768 entitled “COMPOSITIONS AND METHODS OF USING CYANINE DYES TO TARGET CANCER AND MITOCHONDRIA,” filed Jan. 30, 2020, the disclosure of which is incorporated herein by reference in its entirety.

### STATEMENT REGARDING FEDERALLY SPONSORED RESEARCH

This invention was made with government support under CA212600 awarded by the National Institutes of Health. The government has certain rights in this invention.

### BACKGROUND

Cancer is still one of the deadliest threats to human health. In 2012, there were 14 million new cases of cancer worldwide and 8.2 million cancer-related deaths. The number of new cancer cases is expected to rise to 22 million by 2030, and worldwide cancer deaths are projected to increase by 60%. Among men, the five most common sites of cancer diagnosed in 2012 were the lung (16.7% of the total), prostate (15.0%), colorectum (10.0%), stomach (8.5%), and liver (7.5%). Among women, the five most common incident sites of cancer were the breast (25.2% of the total), colorectum (9.2%), lung (8.7%), cervix (7.9%), and stomach (4.8%). Thus, there remains a need in the field for new cancer therapies. The present disclosure addresses this need.

### BRIEF SUMMARY OF THE DISCLOSURE

In certain embodiments, a method of treating, ameliorating, and/or preventing a cancer in a subject is provided. The method includes administering to the subject a composition comprising a therapeutically effective amount of a compound of Formula I, a pharmaceutically acceptable salt thereof, or a geometric isomer thereof:

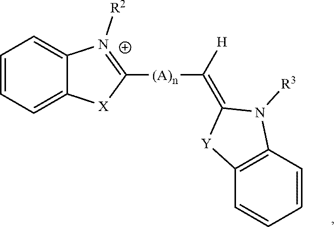

wherein:
X and Y are each independently selected from the group consisting of O, S, CH₂, and C(CH₃)₂;
A is

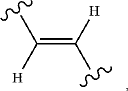

wherein one hydrogen atom in (A)ₙ is optionally substituted by R¹;
R¹ is selected from the group consisting of -C₁₋₅ hydrocarbyl, -C₁₋₅-C(=O)OR, and - C₁-₅ alkoxy;
R₂ and R₃ are each independently selected from the group consisting of -C₁-₅ hydrocarbyl, -C₁₋₅-C(=O)OR, and -C₁₋₅ alkoxy;
each occurrence of R is independently H, C₁₋₄ alkyl, or C₆₋₁₀ aryl; and
n is 0, 1, 2, or 3.

Also provided, in some embodiments, are compounds of Formula I, a pharmaceutically acceptable salt thereof, or a geometric isomer thereof:

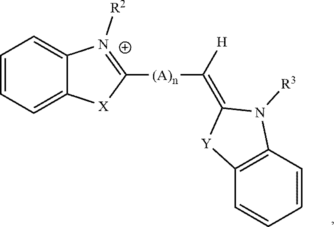

wherein:
X and Y are each independently selected from the group consisting of O, S, CH₂, and C(CH₃)₂;
A is

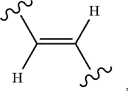

wherein one hydrogen atom in (A)ₙ is optionally substituted by R¹;
R¹ is selected from the group consisting of H, -C₁₋₅ hydrocarbyl, -C₁₋₅-C(=O)OR, and -C₁₋₅ alkoxy;
R₂ and R₃ are each independently selected from the group consisting of -C₁₋₅ hydrocarbyl, -C₁₋₅-C(=O)OR, and -C₁₋₅ alkoxy;
each occurrence of R is independently H, C₁₋₄ alkyl, or C₆₋₁₀ aryl; and
n is 0, 1, 2, or 3,
with the proviso that the compound is not:

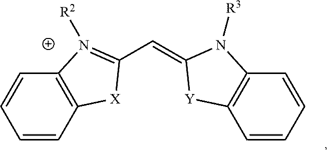

X and Y are S and R² and R³ are methyl or ethyl;

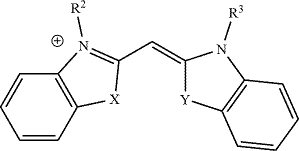

X and Y are O and R² and R³ are ethyl;

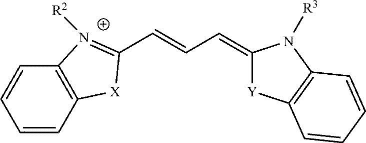

X and Y are S, and R² and R³ are identical and are C₁₋₆ hydrocarbons;

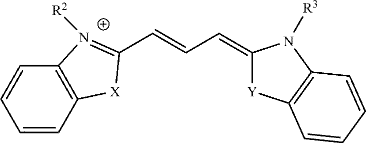

X and Y are O, and R² and R³ are identical and are C₁₋₆ hydrocarbons;

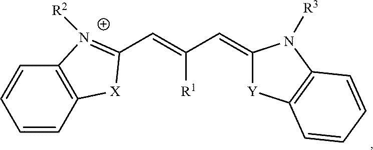

X and Y are S, R¹ is methyl, and R² and R³ are methyl, ethyl or butyl;

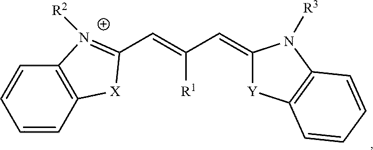

X and Y are S, R¹ is ethyl, and R² and R³ are identical and are C₁₋₄ hydrocarbons;

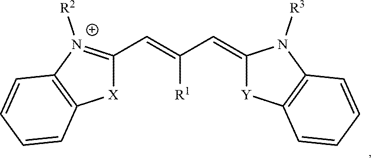

X and Y are O, R¹ is H, and R² and R³ are identical and are C₁₋₆ hydrocarbons;

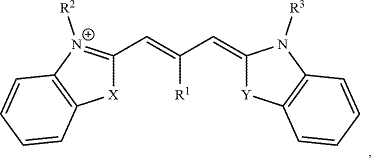

X and Y are O, R¹ is methyl, and R² and R³ are ethyl;

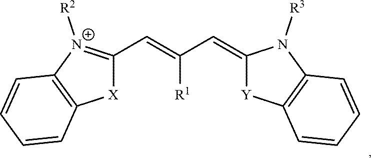

X and Y are O, R¹ is ethyl, and R² and R³ are identical and are methyl or ethyl;

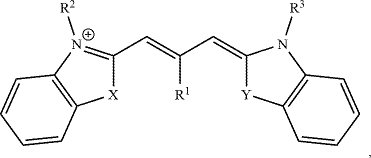

X and Y are C(CH₃)₂, R¹ is H, and R² and R³ are identical and are C₁₋₆ hydrocarbons;

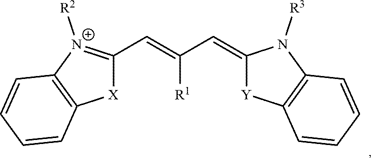

X and Y are C(CH₃)₂, R¹ is methyl, and R² and R³ are ethyl;

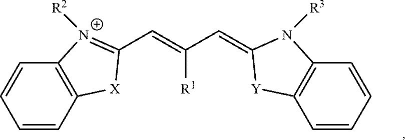

X and Y are C(CH₃)₂, R¹ is ethyl, and R² and R³ are identical and are methyl or ethyl;

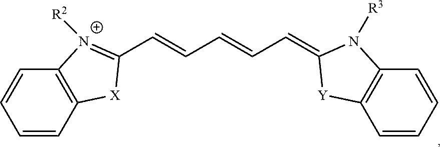

X and Y are S, and R² and R³ are identical and are C₁₋₅ hydrocarbons;

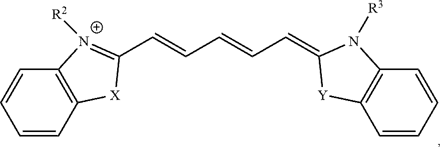

X and Y are O, and R² and R³ are identical and are ethyl, propyl, or pentyl;

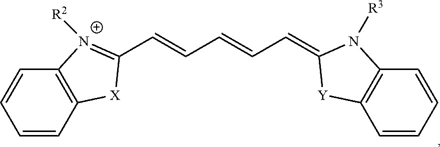

X and Y are C(CH₃)₂, and R² and R³ are identical and are methyl, ethyl, or propyl;

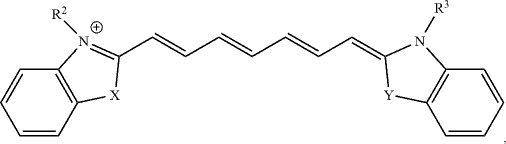

X and Y are O, and R² and R³ are identical and are methyl or ethyl;

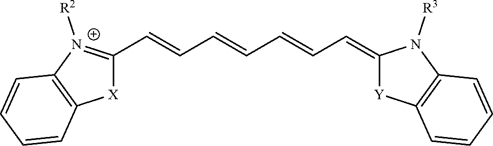

X and Y are S, and R² and R³ are identical and are C₁₋₅ hydrocarbons; or

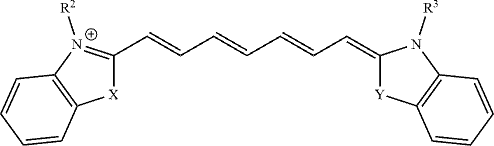

X and Y are C(CH₃)₂, and R² and R³ are identical and are methyl or ethyl.

In certain embodiments, a method of treating, ameliorating, and/or preventing a cancer in a subject is provided. The method includes administering to the subject a composition comprising a therapeutically effective amount of a compound of Formula II, as set forth herein.

### BRIEF DESCRIPTION OF THE FIGURES

The drawings illustrate generally, by way of example, but not by way of limitation, various embodiments of the present application.

FIG. 1 shows viability of cancer cell lines when exposed to CDI (JAC-B2, also known as dithiazine iodide). Cell lines were plated at 1 thousand cells per well and treated with drug for 5 days. Viability was assessed using pico green and IC_{50S} were calculated using graphpad normalized nonlinear regression variable slope.

FIG. 2A shows viability of cancer cell lines when exposed to various cyanine dyes. MAI-PaCa2 cells were plated at 1,000 cells per well in a 96 well plate and treated with varying concentration of the indicated drugs, and with either 25 mM Glucose (RG) or 2 mM Glucose (LG). After five days we analyzed cell survival using Pico Green and determined that the IC_{50S} (µM).

FIG. 2B shows a table with IC50 values of several of the described embodiments in MIA-PaCa2 cells in normal glucose and low glucose conditions.

FIG. 3 shows inhibition of ATP production in mitochondria in a MIA-PaCa2 panceratic cancer cell line by various cyanine dyes. MIA-PaCa2 cells were seeded at 5,000 cells per well, and pretreated for 24 hours with no glucose no FBS media. They were treated with Oligomycin 10 µM (mitochondrial inhibitor), or novel compounds at 0.3 µM each for the novel compounds for 1 hour and ATP was assessed using Cell-Titer Glo.

FIG. 4 shows inhibition of ATP production in mitochondria in a MIA-PaCa2 panceratic cancer cell line by various cyanine dyes. MIA-PaCa2 cells were seeded at 5,000 cells per well, and pretreated for 24 hours with no glucose no FBS media. They were treated with the indicated compounds for 2 hours ATP was assessed using Cell-Titer Glo and IC₅₀’s (nM) were calculated using graphpad.

FIG. 5 shows inhibition of ATP production in mitochondria in a HUH7 liver cancer cell line by various cyanine dyes. HUH7 cells were seeded at 5,000 cells per well, and pretreated for 24 hours with no glucose no FBS media. They were treated with the indicated compounds for 2 hours ATP was assessed using Cell-Titer Glo and IC₅₀’s (nM) were calculated using graphpad.

FIG. 6 shows viability of pancreatic cancer cells (MIA-PaCa2) and lung fibroblast (MRC5) cells after exposure to various doses of cyanine dyes. Cells were seeded at 20,000 cells per well in 96 well plates and treated with varying concentration of the indicated drugs, and with either 25 mM Glucose (RG) or 2 mM Glucose (LG). After two days we analyzed cell survival using Pico Green and determined the IC_{50S} (µM). This figure shows that the compound preferentially target cancer cells compared with fibroblasts.

FIGS. 7A-7S show the effects of combinational therapy of JAC-B2 or JAC-R2 coupled with various chemotherapies and anti-cancer drugs in MIA-PaCa2 cancer cells (7A-7L, 7Q-7S), HT29 cancer cells (70) and ASPC1 cancer cells (7P). Cell lines were plated at 1 thousand cells per well and treated with JAC-B2 or JAC-R2 and another drug for 5 days. We analyzed cell survival using Pico Green and determined the combinational effects through BLISS/HSA plot analyses. (7A) JAC-B2/Gemcitabine ; (7B) JAC-B2/Methotrexate; (7C) JAC-B2/Irinotecan; (7D) JAC-B2/Olaparib; (7E) JAC-B2/Everolimus; (7F) JAC-B2/Paclitaxel; (7G) JAC-B2/Oxaliplatin; (7H) JAC-B2/Abemaciclib. (7I) JAC-B2/Decitabine; (7J) JAC-B2/Azacitidine; (7K) JAC-R2/Decitabine; (7L) JAC-R2/Azacitidine; (7M) JAC-R2/Paclitaxel; (7N) JAC-B2/5-Fluorouracil; (70) JAC-B2/Cobimetinib; (7P) JAC-B2/PDDX04; (7Q) JAC-B2/Colchicine; (7R) JAC-B2/Adavosertib; (7S) JAC-B2/VE-821. The circled regions in FIGS. 7A and 7D are regions of antagonistic response.

FIG. 8 shows a schematic for determining the suitability of treating cancer in a subject with a compound of Formula I.

FIG. 9 shows JAC-B2 IC₅₀ values (expressed in -Log₁₀(IC₅₀) in 59 cancer cell lines from the NIC-60 cancer cell lines dataset.

FIG. 10A shows an example of two significant inversely correlating genes, in which increased gene expression levels correlated with increased resistance (higher IC_{50S}) to JAC-B2 across the NCI-60 dataset. This supports the rationale that tumor response to JAC-B2 and other analogs could potentially be predicted based of molecular data.

FIG. 10B shows an example of two significant positively correlating genes, in which increased gene expression levels correlated with increased susceptibility (lower IC_{50S}) to JAC-B2 across the NCI-60 dataset. This supports the rationale that tumor response to JAC-B2 and other analogs could potentially be predicted based of molecular data.

FIG. 11 shows a possible response prediction model in which, after analyzing the top 10 positive and top 10 negative correlating genes, a regression analysis found two gene -MYO1B and CPT1A to be strongly significant and independent (P<0.05). The figure shows the actual and predicted -Log₁₀(IC₅₀) of JAC-B2 across the NCI-60 cancer cell line set.

FIG. 12 shows the application of the model described in FIG. 11 on 1036 cell lines from the cancer cell line encyclopedia (CCLE) showing potential use for JAC-B2 in hematological, brain, gastrointestinal and gynecological malignancies.

FIG. 13 shows an example of two significant inversely correlating hallmark gene set signatures, in which increased gene expression levels correlated with increased resistance (higher IC_{50S}) to JAC-B2 across the NCI-60 dataset. This supports the rationale that tumor response to JAC-B2 and other analogs could potentially be predicted based on signatures of key cellular pathways or processes.

FIG. 14 shows an example of top mitochondrial gene set signatures significantly correlating increased resistance (higher IC_{50S}) to JAC-B2 across the NCI-60 dataset. This supports the rationale that tumor response to JAC-B2 and other analogs could potentially be predicted based on signatures of key mitochondrial pathways or processes.

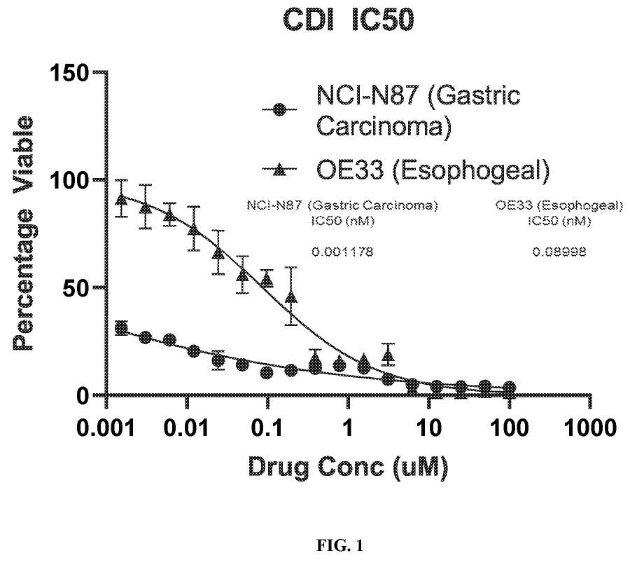

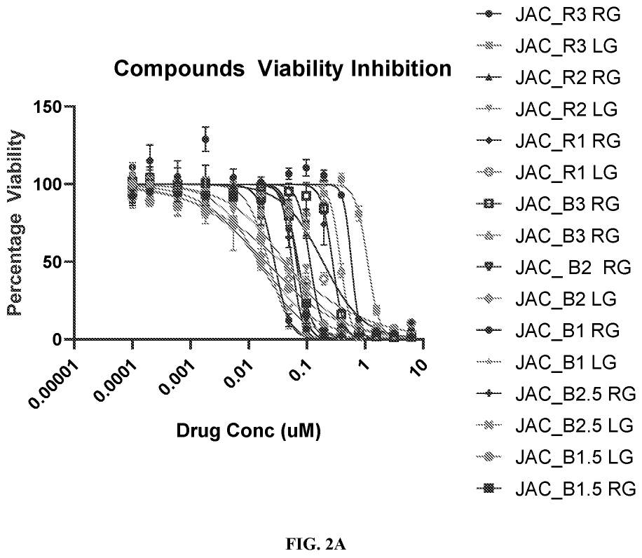

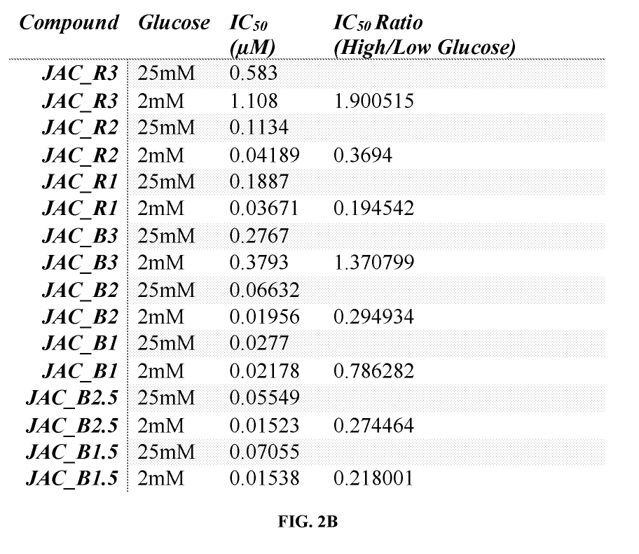

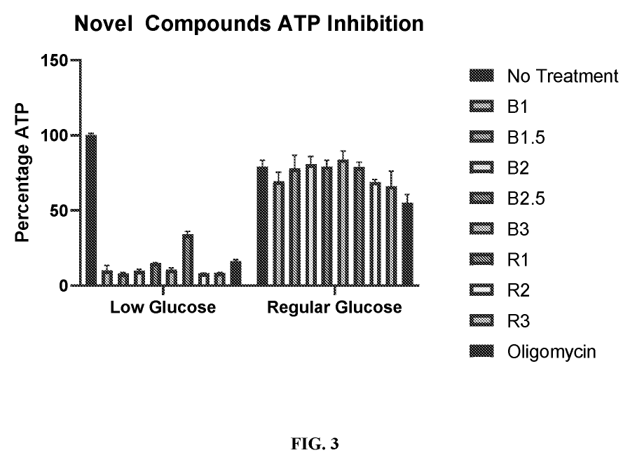

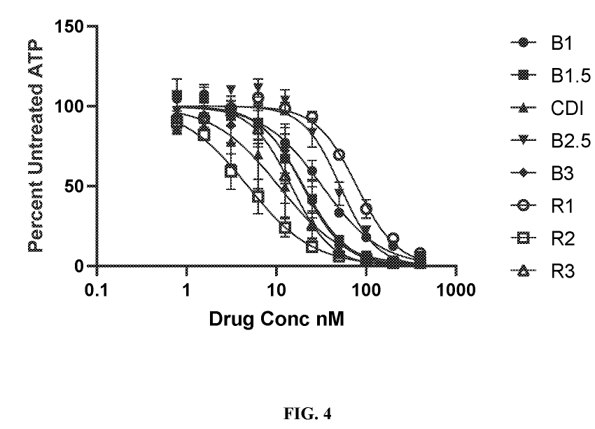

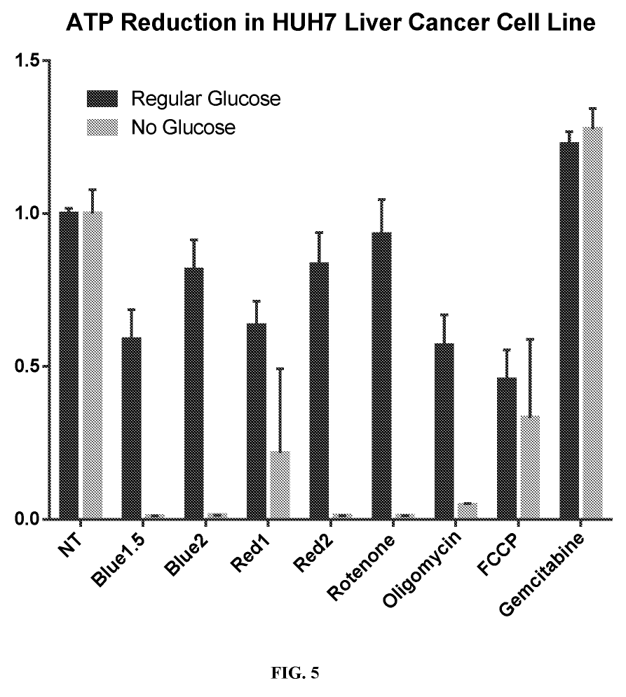

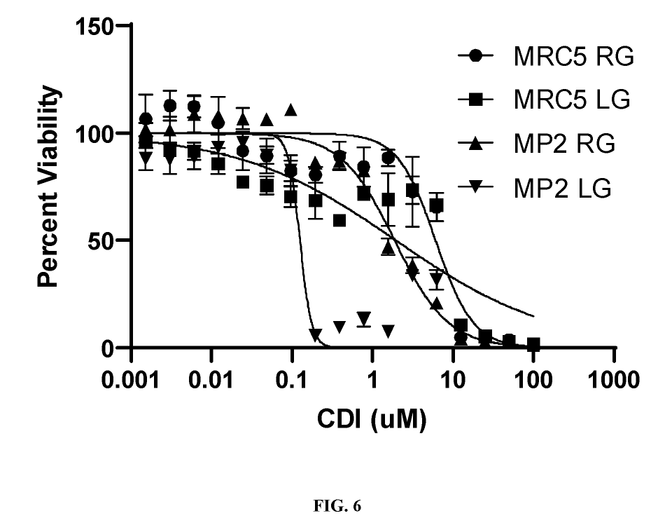

### DETAILED DESCRIPTION OF THE DISCLOSURE

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less. The term “substantially free of” can mean having a trivial amount of, such that a composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less, or about 0 wt%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂, C(O)N(OR)R, C(=NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂, C(O)N(OR)R, and C(=NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, -CH=C=CCH₂, -CH=CH(CH₃), -CH=C(CH₃)₂, -C(CH₃)=CH₂, -C(CH₃)=CH(CH₃), -C(CH₂CH₃)=CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to -C≡CH, -C≡C(CH₃), -C≡C(CH₂CH₃), -CH₂C≡CH, -CH₂C≡C(CH₃), and -CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl) , indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3- pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R-NH2, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form -NH₂, -NHR, -NR₂, -NR₃⁺, wherein each R is independently selected, and protonated forms of each, except for -NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, polyhalo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal’s health continues to deteriorate.

In contrast, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer’s solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington’s Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Preparation of Compounds

Compounds of Formula I or Formula II or otherwise described herein can be prepared by the general schemes described herein, using the synthetic method known by those skilled in the art. The following examples illustrate non-limiting embodiments of the compound(s) described herein and their preparation.

In various embodiments, a compound of Formula I, a pharmaceutically acceptable salt thereof, or a geometric isomer thereof has the structure:

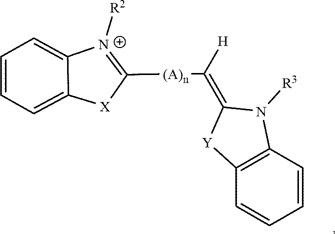

In the compound of Formula I,
X and Y are each independently selected from the group consisting of O, S, CH₂, and C(CH₃)₂;
A is

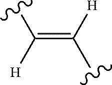

(trans double bond), wherein one hydrogen atom in (A)ₙ is optionally substituted by R¹;
R¹ is selected from the group consisting of H, -C₁₋₅ hydrocarbyl, -C₁₋₅-C(=O)OR, and -C₁₋₅ alkoxy;
R₂ and R₃ are each independently selected from the group consisting of -C₁₋₅ hydrocarbyl, -C₁₋₅-C(=O)OR, and -C₁₋₅ alkoxy;
each occurrence of R is independently H, C₁₋₄ alkyl, or C₆₋₁₀ aryl; and
n is 0, 1, 2, or 3.

In some embodiments, A is

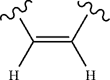

(cis double bond), wherein one hydrogen atom in (A)ₙ is optionally substituted by R¹. Variable ‘A’ can contain any combination of cis and trans double bonds, and optionally contains all cis double bonds or all trans double bonds.

In various embodiments, the compound is not:

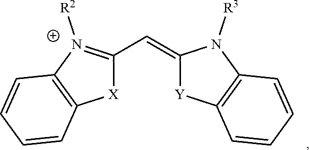

X and Y are S and R² and R³ are methyl or ethyl;

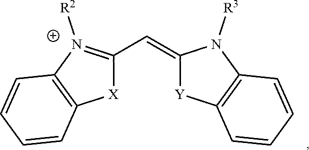

X and Y are O and R² and R³ are ethyl;

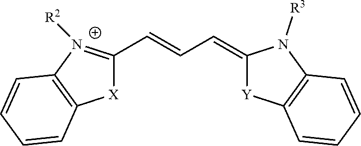

X and Y are S, and R² and R³ are identical and are C₁₋₆ hydrocarbons;

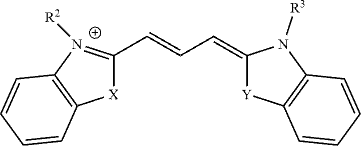

X and Y are O, and R² and R³ are identical and are C₁₋₆ hydrocarbons;

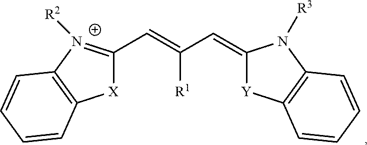

X and Y are S, R¹ is methyl, and R² and R³ are methyl, ethyl, or butyl;

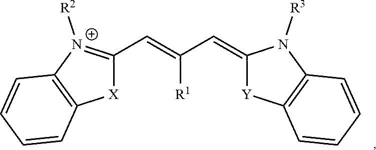

X and Y are S, R¹ is ethyl, and R² and R³ are identical and are C₁₋₄ hydrocarbons;

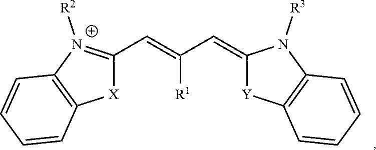

X and Y are O, R¹ is H, and R² and R³ are identical and are C₁₋₆ hydrocarbons;

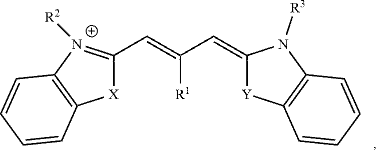

X and Y are O, R¹ is methyl, and R² and R³ are ethyl;

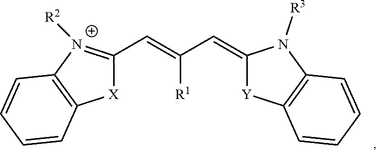

X and Y are O, R¹ is ethyl, and R² and R³ are identical and are methyl or ethyl;

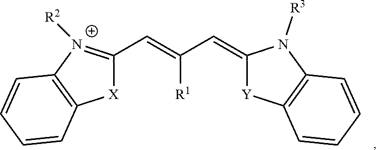

X and Y are C(CH₃)₂, R¹ is H, and R² and R³ are identical and are C₁₋₆ hydrocarbons;

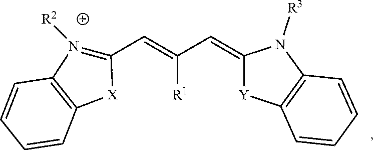

X and Y are C(CH₃)₂, R¹ is methyl, and R² and R³ are ethyl;

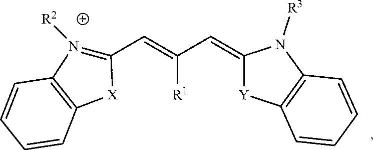

X and Y are C(CH₃)₂, R¹ is ethyl, and R² and R³ are identical and are methyl or ethyl;

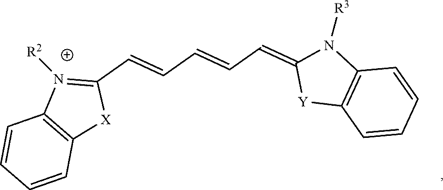

X and Y are S, and R² and R³ are identical and are C₁₋₅ hydrocarbons;

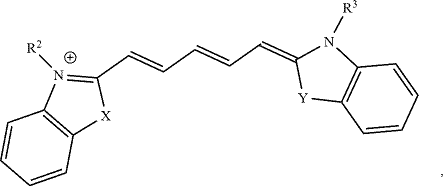

X and Y are O, and R² and R³ are identical and are ethyl, propyl, or pentyl;

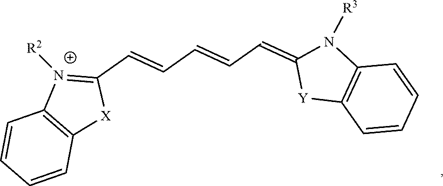

X and Y are C(CH₃)₂, and R² and R³ are identical and are methyl, ethyl, or propyl;

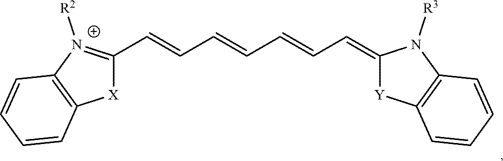

X and Y are O, and R² and R³ are identical and are methyl or ethyl;

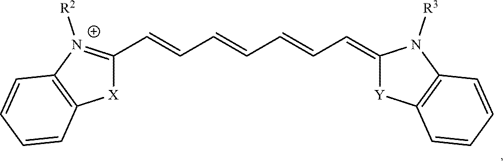

X and Y are S, and R² and R³ are identical and are C₁₋₅ hydrocarbons; or

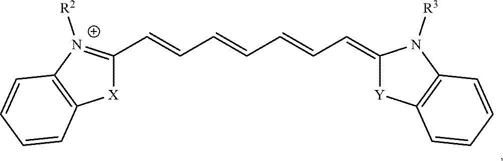

X and Y are C(CH₃)₂, and R² and R³ are identical and are methyl or ethyl.

The wavy lines in A,

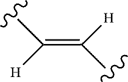

, represent attachment points for the rest of the molecule in Formula I or points where additional vinyl groups are attached. When is n is 0, A is a bond (absent). When n is 1, 2, or 3, (A)ₙ is

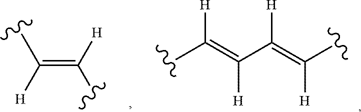

or

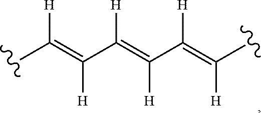

respectively. Optionally, one hydrogen atom in (A)ₙ is substituted by R¹, as defined herein. When R¹ is hydrogen, (A)ₙ is unsubstituted.

In various embodiments, the pharmaceutically acceptable salt is a halide salt, such as fluoride, chloride, bromide, or iodide. In certain embodiments, the pharmaceutically acceptable salt is iodide. In certain embodiments, R¹ is H. In some embodiments, R² and R³ are -C₁₋₅-C(=O)OR. In some embodiments, R² and R³ are -(CH₂)₄-C(=O)OR. In certain embodiments, n is 0.

The compound of Formula Ia, a pharmaceutically acceptable salt thereof, or a geometric isomer thereof, in a certain embodiment has the structure:

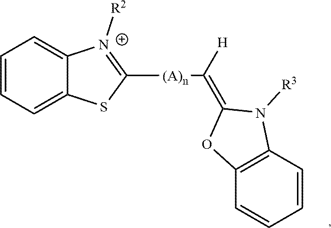

where the variables R¹, R², R³, A, and n are as defined herein. In certain embodiments, in the compound of Formula Ia, R² and R³ are ethyl.

In various embodiments, the compound is selected from the group consisting of

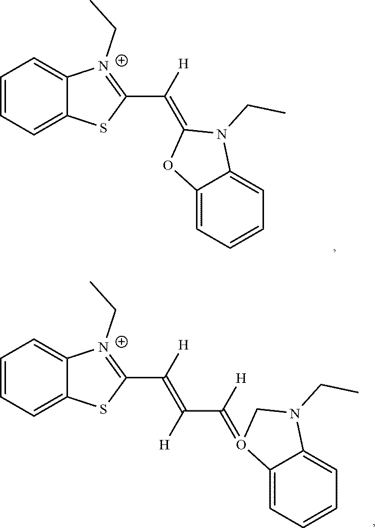

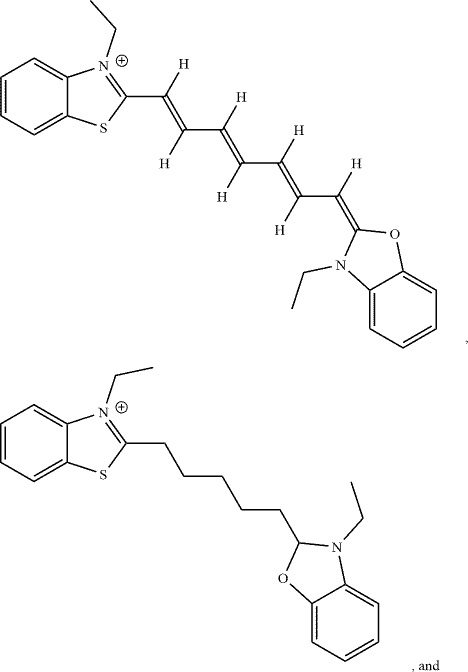

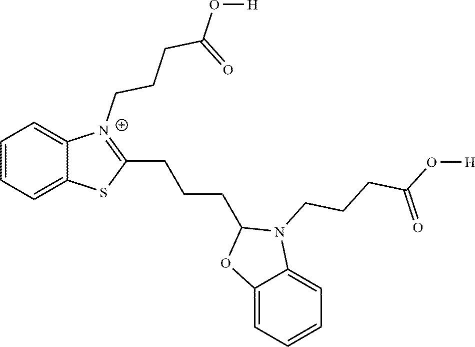 [SMILES: O=C(O)CCCN1c2ccccc2OC1CCCc1sc2ccccc2[n+]1CCCC(=O)O]

In various embodiments, a compound of Formula II, a pharmaceutically acceptable salt thereof, or a geometric isomer thereof has the structure:

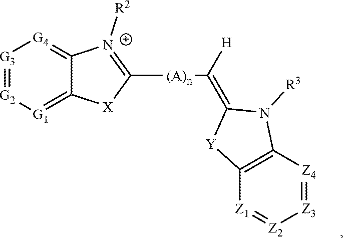

In the compound of Formula II,
each of G₁, G₂, G₃, and G₄ is independently C(=O), CR′, CNR″₂, N, or N-R″;
each of Z₁, Z₂, Z₃, and Z₄ is independently C(=O), CR′, CNR″₂, N, or N-R″;
X and Y are each independently selected from the group consisting of O, N, S, CH₂, and C(CH₃)₂;
A is

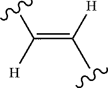

(trans double bond), wherein one hydrogen atom in (A)ₙ is optionally substituted by R¹;
R¹ is selected from the group consisting of H, -C₁₋₅ hydrocarbyl, -C₁₋₅-C(=O)OR, and -C₁₋₅ alkoxy;
R₂ and R₃ are each independently selected from the group consisting of -C₁₋₅ hydrocarbyl, -C₁₋₅-C(=O)OR, and -C₁₋₅ alkoxy;
each occurrence of R′ is independently H or C₁₋₄ alkyl;
each occurrence of R″ is independently H or C₁₋₄ alkyl;
each occurrence of R is independently H, C₁₋₄ alkyl, or C₆₋₁₀ aryl; and
n is 0, 1, 2, or 3.

In some embodiments, A is

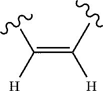

(cis double bond), wherein one hydrogen atom in (A)ₙ is optionally substituted by R¹. Variable ‘A’ can contain any combination of cis and trans double bonds, and optionally contains all cis double bonds or all trans double bonds.

The wavy lines in A,

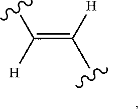

represent attachment points for the rest of the molecule in Formula II or points where additional vinyl groups are attached. When is n is 0, A is a bond (absent). When n is 1, 2, or 3, (A)ₙ is

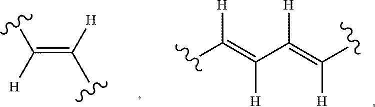

or

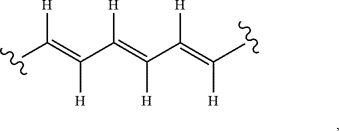

respectively. Optionally, one hydrogen atom in (A)ₙ is substituted by R¹, as defined herein. When R¹ is hydrogen, (A)ₙ is unsubstituted.

In various embodiments, the pharmaceutically acceptable salt is a halide salt, such as fluoride, chloride, bromide, or iodide. In certain embodiments, the pharmaceutically acceptable salt is iodide. In certain embodiments, R¹ is H. In some embodiments, R² and R³ are -C₁₋₅-C(=O)OR. In some embodiments, R² and R³ are -(CH₂)₄-C(=O)OR. In certain embodiments, n is 0.

In the compound of Formula II, variables G₁-G₄ and Z₁-Z₄ are, in some embodiments, selected such that the aromaticity of the ring in which they are present is maintained. The ring containing variables G₁-G₄ can be the same or different from the ring containing variables Z₁-Z₄.

In various embodiments, the compound of Formula II can have a structure selected from the group consisting of:

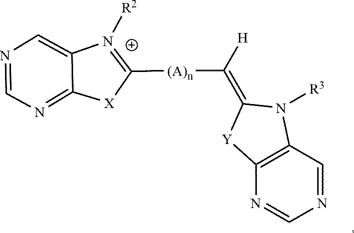

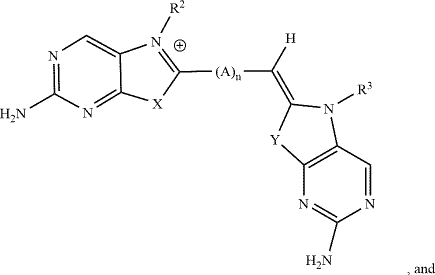

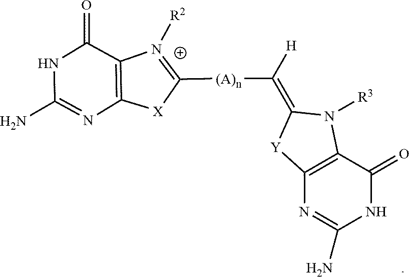

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser’s Reagents for Organic Synthesis, Volumes 1-17 (John Wiley and Sons, 1991); Rodd’s Chemistry of Carbon Compounds, Volumes 1-5 and Supplementals (Elsevier Science Publishers, 1989); Organic Reactions, Volumes 1-40 (John Wiley and Sons, 1991), Larock’s Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while coexisting amino groups are blocked with fluoride labile silyl carbamates.

Allyl blocking groups are useful in the presence of acid- and base- protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

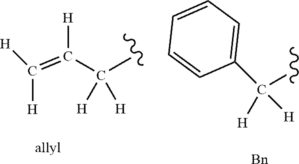

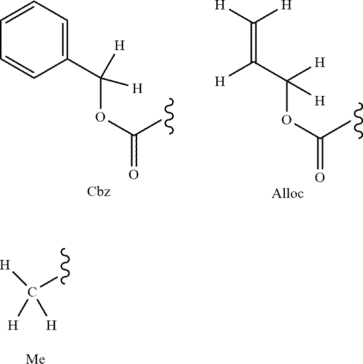

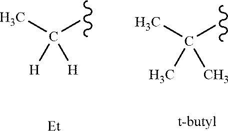

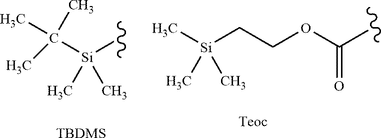

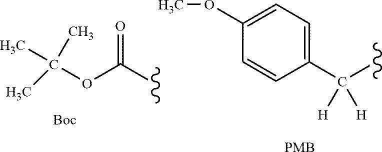

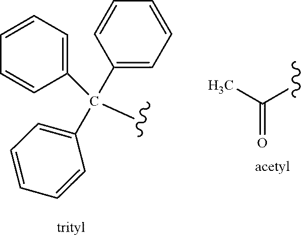

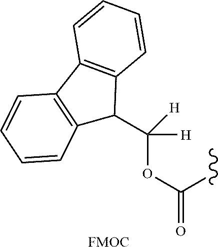

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Pharmacology

In various embodiments, the compound(s) described herein can be administered to a subject in an amount ranging from about 0.01 mg/kg to about 200 mg/kg, or about 0.5 mg/kg to about 190 mg/kg, or about 0.75 mg/kg to about 180 mg/kg, or about 1 mg/kg to about 170 mg/kg, or about 1.5 mg/kg to about 160 mg/kg, or about 2 mg/kg to about 150 mg/kg, or about 2.5 mg/kg to about 140 mg/kg, or about 3 mg/kg to about 130 mg/kg, or about 3.5 mg/kg to about 120 mg/kg, or about 4 mg/kg to about 110 mg/kg, or about 4.5 mg/kg to about 100 mg/kg, or about 5 mg/kg to about 95 mg/kg, or about 5.5 mg/kg to about 90 mg/kg, or about 6 mg/kg to about 85 mg/kg, or about 6.5 mg/kg to about 80 mg/kg, or about 7 mg/kg to about 75 mg/kg, or about 7.5 mg/kg to about 70 mg/kg, or about 8 mg/kg to about 65 mg/kg, or about 8.5 mg/kg to about 60 mg/kg, or about 9 mg/kg to about 55 mg/kg or about 9.5 mg/kg to about 50 mg/kg, or about 10 mg/kg to about 45 mg/kg.

In various embodiments, the compound(s) described herein can be administered to a subject in an amount that is less than, equal to, or greater than about 0.01 mg/kg, 0.05 mg/kg, 0.1 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 100 mg/kg, 105 mg/kg, 110 mg/kg, 115 mg/kg, 120 mg/kg, 125 mg/kg, 130 mg/kg, 140 mg/kg, 145 mg/kg, 150 mg/kg, 155 mg/kg, 160 mg/kg, 170 mg/kg, 175 mg/kg, 180 mg/kg, 185 mg/kg, 190 mg/kg, 195 mg/kg, or 200 mg/kg.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treating Cancer

The disclosure includes a method of treating, ameliorating, and/or preventing cancer using the compounds of Formula I or Formula I. The disclosure also includes a method of preventing, ameliorating, and/or preventing cancer using the compounds of Formula I or Formula II. Non-limiting examples of cancer include liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer, and the like.

In various embodiments, the method of treating, ameliorating, and/or preventing a cancer in a subject in need thereof, includes administering to the subject a composition comprising a therapeutically effective amount of a compound of Formula I or Formula II, a pharmaceutically acceptable salt thereof, or a geometric isomer thereof:

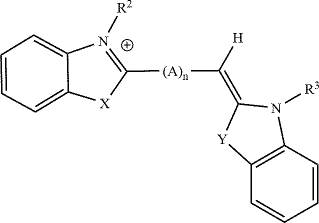

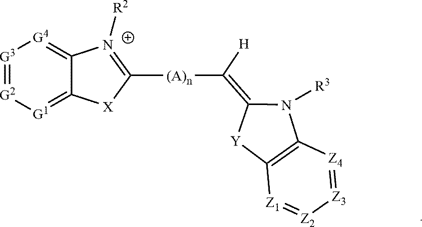

In the method of treating, ameliorating, and/or preventing cancer with a compound of Formula I, the variables R¹, R², R³, A, and n are as defined herein.

In the method of treating, ameliorating, and/or preventing cancer with a compound of Formula II, the variables R¹, R², R³, A, G₁-G₄, Z₁-Z₄, and n are as defined herein.

In various embodiments, the pharmaceutically acceptable salt is a halide salt. In various embodiments, the pharmaceutically acceptable salt is iodide.

In certain embodiments, the composition includes an encapsulating agent. Methods of formulating compounds for the treatment of cancer in encapsulating agents are known by those of skill in the art. The encapsulating agent is a biocompatible structure that substantially or entirely envelops a compound of Formula I of Formula II. In some embodiments, the encapsulating agent includes a nanoparticle or a liposome. In certain embodiments, the encapsulating agent includes a micelle, a nucleic-acid based dendrimer nanocarrier, a protein-based nanocarrier, a polymer-based nanoparticle, a lipid-polymer based nanoparticle, a metal-based nanoparticle, a nanocrystal, a liposomal carrier, a carbon nanotube based nanoparticle, or a polymeric micelle. In some embodiments, the encapsulating agent comprises mammalian serum albumin. The mammalian serum albumin is, in some embodiments, of human origin.

The encapsulating agent can further include a targeting agent. The targeting agent is a biocompatible substance that directs or facilitates the exposure of the compound of Formula I or Formula II to the target cancer. The target cancer includes cancer cells in primary tumors, cancer cells in the target organ, and/or cancer cells present throughout the body of the subject.

In some embodiments, targeting agent includes a peptide ligand, a nucleotide ligand, a polysaccharide ligand, a fatty acid ligand, a lipid ligand, a small molecule ligand, an antibody (such as a MUC1-antibody), an antibody fragment, an antibody mimetic, or an antibody mimetic fragment. In certain embodiments, the targeting agent includes folic acid or transferrin. In some embodiments, the composition is administered orally or parenterally.

In certain embodiments, the compound is not

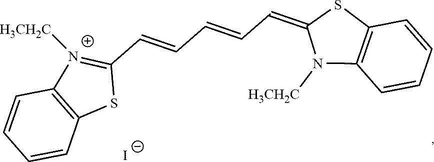

and the cancer is selected from group consisting of liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer.

In another embodiment, the compound is

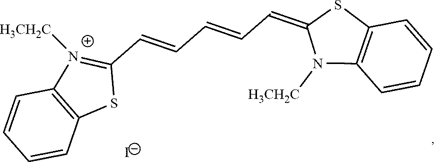

and the cancer is selected from the group consisting of liver cancer, rectal cancer, gastric cancer, esophageal cancer, anal cancer, non-melanoma skin cancer, small intestine cancer, bile duct cancer, periampullary cancer, endometrial cancer, cervical cancer, small cell lung cancer, sarcoma, neuroendocrine cancer, lymphoma, hepatoblastoma, Wilm’s tumor and primary mesothelial cancer.

In certain embodiments, the composition is administered at an advanced stage of peritoneal involvement by a cancer selected from the group consisting of liver cancer, rectal cancer, gastric cancer, esophageal cancer, head& neck cancer, renal cancer, breast cancer, ovarian cancer, endometrial cancer, cervical cancer, anal cancer, skin cancer, prostate cancer, small intestine cancer, bile duct cancer, pancreatic cancer, lung cancer, sarcoma, neuroendocrine cancer, and primary mesothelial cancer.

In various embodiments, the neuroendocrine cancer is selected from the group consisting of adrenocortical carcinoma, a carcinoid tumor, and thymic cancer.

In certain embodiments, the method includes administering at least one other chemotherapeutic agent. The at least one other chemotherapeutic agent can include a DNA damaging agent, a microtubule inhibitor, a Topoisomerase inhibitor, an anti-metabolite , a cell cycle checkpoint inhibitor, a mechanistic target of rapamycin kinase (mTOR) inhibitor, a receptor tyrosine kinase (RTK) inhibitor, a DNA damage repair inhibitor, an anti-cell surface protein, an immune checkpoint inhibitor, an immune modulator, a DNA methyltransferase inhibitor, or a combination chemotherapy, and the like.

The DNA damaging agent can include 5-Fluorouracil, Gemcitabine, Cytarabine, Capecitabine, Dacarbazine, Temozolomide, Mitomycin C, Paclitaxel, Docetaxel, Ifosfamide, Cyclophosphamide, Adriamycin, Doxorubicin, Cisplatin, Carboplatin, or Oxaliplatin, and the like. The microtubule inhibitor can include Vincristine, Vinorelbine, or Vinblastine, and the like. The topoisomerase inhibitor can include Topotecan, Irinotecan, or Etoposide, and the like. The CDK inhibitor can include an inhibitor of CDK4, an inhibitor of CDK6 or an inhibitor of CDK4 and CDK6. The DNA damage repair inhibitor can include Olaparib, Veliparib, or Talazoparib, and the like. The DNA methyltransferase inhibitor can include Decitabine or Azacitidine.

In certain embodiments, the method further includes administering at least one hormone antagonist, selective hormone receptor modulating agent, or hormone enzyme inhibitor. In certain embodiments, the method further includes radiation therapy. In certain embodiments, the method of treatment alleviates a symptom of the cancer. In certain embodiments, the subject is human.

In various embodiments, the subject has a plasma compound concentration about 0.2 ng/mL to about 20 ng/mL at about 1 hour after administration of the composition. In various embodiments, the subject has a plasma compound concentration of about 0.5 ng/mL to about 50 ng/mL at about 4 hours after administration of the composition. In various embodiments, the subject has a plasma compound concentration of about 0.5 ng/mL to about 50 ng/mL at up to about 5 hours after administration of the composition. In various embodiments, the subject has a compound in the pancreas of about 5 ng/mL to about 100 ng/mL at about 4 hours after administration of the composition. In various embodiments, the subject has a compound concentration in the pancreas of about 1 ng/mL to about 100 ng/mL at about 8 hours after administration of the composition. In various embodiments, the subject has a compound concentration in the pancreas of about 100 ng/mL to about 400 ng/mL at up to about 8 hours after administration of the composition.

In some embodiments, the subject has failed at least one, at least two, or at least three prior cancer therapies (e.g. chemotherapy, radiation, immunotherapy) prior to administration of a compound of Formula I.

In some embodiments, the subject has failed at least one, at least two, or at least three prior cancer therapies (e.g. chemotherapy, radiation, immunotherapy) prior to administration of a compound of Formula II.

| | |
|---|---|
| Compound (iodide salt) | Common Name (Identifier) |
| 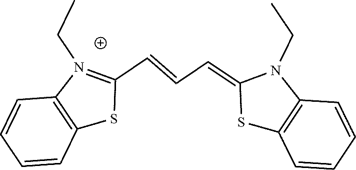 [SMILES: CCN1/C(=C/C=C/c2sc3ccccc3[n+]2CC)Sc2ccccc21] | 3′,3′-Diethylthiacarbocyanine iodide (JAC-B1) |
| 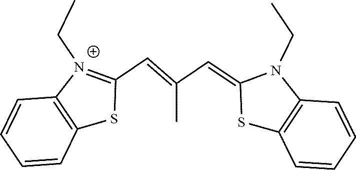 [SMILES: CCN1/C(=C/C(C)=C/c2sc3ccccc3[n+]2CC)Sc2ccccc21] | 3,3′-Diethyl-9-methylthiacarbocyanine iodide (JAC-B2.5) |
| 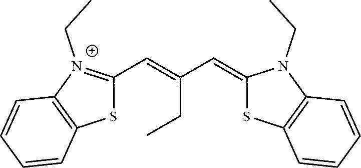 [SMILES: CCC(/C=C1\Sc2ccccc2N1CC)=C\c1sc2ccccc2[n+]1CC] | 3,3′-Dimethyl-9-Ethylthiacarbocyanine iodide (JAC-B1.2) |
| 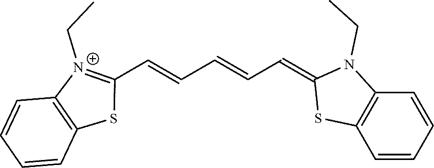 [SMILES: CCN1/C(=C/C=C/C=C/c2sc3ccccc3[n+]2CC)Sc2ccccc21] | Dithiazanine Iodide (JAC-B2 or CDI) |
| 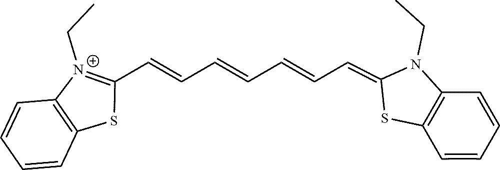 [SMILES: CCN1/C(=C/C=C/C=C/C=C/c2sc3ccccc3[n+]2CC)Sc2ccccc21] | 3′, 3′-Diethylthiatricarbocanine iodide (JAC-B3) |
| 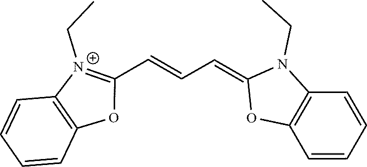 [SMILES: CCN1/C(=C/C=C/c2oc3ccccc3[n+]2CC)Oc2ccccc21] | 3′,3′-Diethyloxacarbocyanine iodide (JAC-R1) |
| 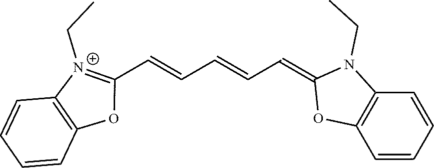 [SMILES: CCN1/C(=C/C=C/C=C/c2oc3ccccc3[n+]2CC)Oc2ccccc21] | 3′,3′-Diethyloxadicarbocyanine iodide (JAC-R2) |
| 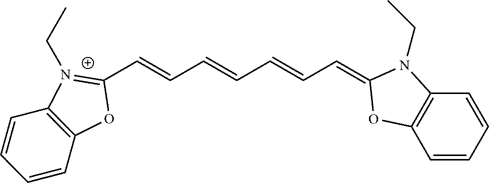 [SMILES: CCN1/C(=C/C=C/C=C/C=C/c2oc3ccccc3[n+]2CC)Oc2ccccc21] | 3′,3′-Diethyloxatricarbocyanine iodide (JAC-R3) |

Without being bound by theory, the compounds of Formula I or Formula II are believed to inhibit mitochondria in cancerous cells. In various embodiments, the compounds of Formula I or Formula II preferentially target hypoglycemic (low glucose) microenvironments. Hypoglycemic environments in cancerous cells frequently occur in cancers such as pancreatic cancer, peritoneal carcinomatosis, sarcomas, and the like. In some embodiments, the compounds of Formula I or Formula II selectively target cancerous tissue, such as tumors, that contain hypoglycemic microenvironments over tissue that has normal or elevated levels of cellular glucose. Similarly in some embodiments the compounds of Formula I or Formula II are given in combination with other metabolically active drugs to further disrupt the cellular energy production. For example, the compounds of Formula I or Formula II can be given with 2-deoxy-glucose (a competitor of glucose) in order to mimic a hypoglycemic state in the cells thereby rendering the compounds to preferentially target cancer cells.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats cancer.

In certain embodiments, administering the compound(s) described herein to the subject allows for administering a lower dose of the additional therapeutic agent as compared to the dose of the additional therapeutic agent alone that is required to achieve similar results in treating cancer in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Method of Treating Cancer Based on Predicting of Response to Treatment with Compounds of Formula I or Formula II

In various embodiments, a method of predicting whether a patient will favorable respond to treatment of cancer with one or more compound of Formula I or Formula II is provided. The method includes obtaining the results of a correlation between a subject biological profile information and the predictive information of post-treatment efficacy for a particular treatment selected from one or more treatments for a cancer using one or more compounds of Formula I or Formula II. The subject’s biological profile can be obtained from one or more tissue samples provided by the subject such as, for example, a tumor biopsy. The tissue sample can contain a plurality of cancerous cells that can be analyzed and profiled to determine whether treatment with compounds of Formula I or Formula II is likely to be efficacious.

The method further includes identifying at least one gene, protein or cellular genetic pathway from the expression levels that is involved in key metabolic or mitochondrial activities, creating a regression model derived from experimental activity data of the compound of Formula I or Formula II on least one cellular molecular or pathway target in cancer tissue, measuring expression levels from the at least one cellular genetic pathway in a tissue sample obtained from the subject, and treating the subject with the compound of Formula I or Formula II if the regression model predicts a potency of the compound of Formula I or Formula II on the tissue sample above a predetermined threshold level.

In various embodiments, the predetermined threshold level is a predicted -log(IC₅₀) of at least 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In various embodiments, the predetermined threshold level is a predicted -log(IC₅₀) of at least 5.0. The regression model can be, in some embodiments, a linear regression model.

In various embodiments, the cells from the tissue sample are profiled using known cellular profiling methods that measure gene expression, genetic RNA signatures, protein levels, signaling molecule levels, cytokine levels, or combinations thereof, in the cells. In various embodiments, the biological profile includes a plurality of mitochondrial signatures from the cells in the tissue sample. The cellular profiling includes exposing a plurality of cells from the tissue sample to one or more compounds of Formula I or Formula II. In certain embodiments, the cells are exposed to JAC-B2.

In various embodiments, a method for predicting cancer drug response in a subject, the method includes obtaining the results of molecular data from a subject’s tissue sample, using a response prediction model on the data and applying results of the model to select a particular treatment for cancer, wherein the particular treatment comprises a treatment with a compound of Formula I or Formula II or a pharmaceutically acceptable salt or geometric isomer thereof, wherein the correlation is obtained by:
i. identifying, based on gene, protein or metabolic expression levels, at least one gene product, protein, metabolite, cellular hallmark pathway, or one cellular genetic pathway involved in mitochondrial activity; and
ii. creating a regression or correlation model derived from experimental activity data of the compound of Formula I or Formula II on the at least one cellular pathway or gene product in the subject’s tissue sample.

The compound of Formula I, in various embodiments, has the structure:

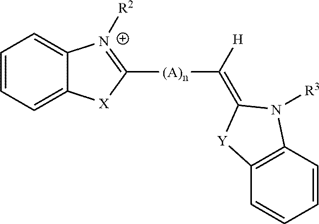

wherein:
X and Y are each independently selected from the group consisting of O, S, CH₂, and C(CH₃)₂;
A is

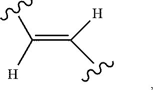

wherein one hydrogen atom in (A)ₙ is optionally substituted by R¹;
R¹ is selected from the group consisting of -C₁₋₅ hydrocarbyl, -C₁₋₅-C(=O)OR, and -C₁₋₅ alkoxy;
R₂ and R₃ are each independently selected from the group consisting of -C₁₋₅ hydrocarbyl, -C₁₋₅-C(=O)OR, and -C₁₋₅ alkoxy;
each occurrence of R is independently H, C₁₋₄ alkyl, or C₆₋₁₀ aryl; and
n is 0, 1, 2, or 3.

The compound of Formula II, in some embodiments, has the structure:

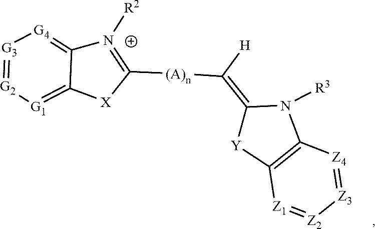

wherein:
each of G₁, G₂, G₃, and G₄ is independently C(=O), CR′, CNR″₂, N, or N-R″;
each of Z₁, Z₂, Z₃, and Z₄ is independently C(=O), CR′, CNR″₂, N, or N-R″;
X and Y are each independently selected from the group consisting of O, N, S, CH₂, and C(CH₃)₂;
A is

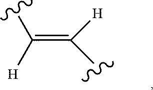

wherein one hydrogen atom in (A)ₙ is optionally substituted by R¹;
R¹ is selected from the group consisting of -C₁₋₅ hydrocarbyl, -C₁₋₅-C(=O)OR, and -C₁₋₅ alkoxy;
R₂ and R₃ are each independently selected from the group consisting of -C₁₋₅ hydrocarbyl, -C₁₋₅-C(=O)OR, and -C₁₋₅ alkoxy;
each occurrence of R′ is independently H or C₁₋₄ alkyl;
each occurrence of R″ is independently H or C₁₋₄ alkyl;
each occurrence of R is independently H, C₁₋₄ alkyl, or C₆₋₁₀ aryl; and
n is 0, 1, 2, or 3.

The method for predicting cancer drug response in a subject can further include, in some embodiments, wherein the treatment response prediction is used to treat cancer in the subject by obtaining molecular data from the subject’s tissue sample; and treating the subject with the compound of Formula I or Formula II if the response model predicts a potency of the compound of Formula I or Formula II on the tissue sample above a predetermined threshold level. In various embodiments, the predetermined threshold level is a predicted -log(IC₅₀) of at least 5.0. In various embodiments, the model is based on experimental activity of at least one compound of Formula I or Formula II on the a gene set containing at least, one gene target or signature from Tables 1A-1C as set forth herein. In various embodiments, the model is based on experimental activity of at least one compound of Formula I on a molecular expression model containing at least the MYO1B and CPT1A genes.

In certain embodiments, RNA-Seq data was analyzed from 59 NCI-60 cell lines. Single Set Gene Set Enrich Analysis (ssGSEA) was employed to gather pathway and set signatures from the GSEA hallmark set and a individually curated set of ~100 mitochondrial signatures. Signatures and Gene expression levels were correlated to cell line IC₅₀ and to -log(IC₅₀) using both Pearson’s and Spearman’s correlation tests. In certain embodiments, the Benjamini & Hochberg method was employed to find the false discovery rate of each set.

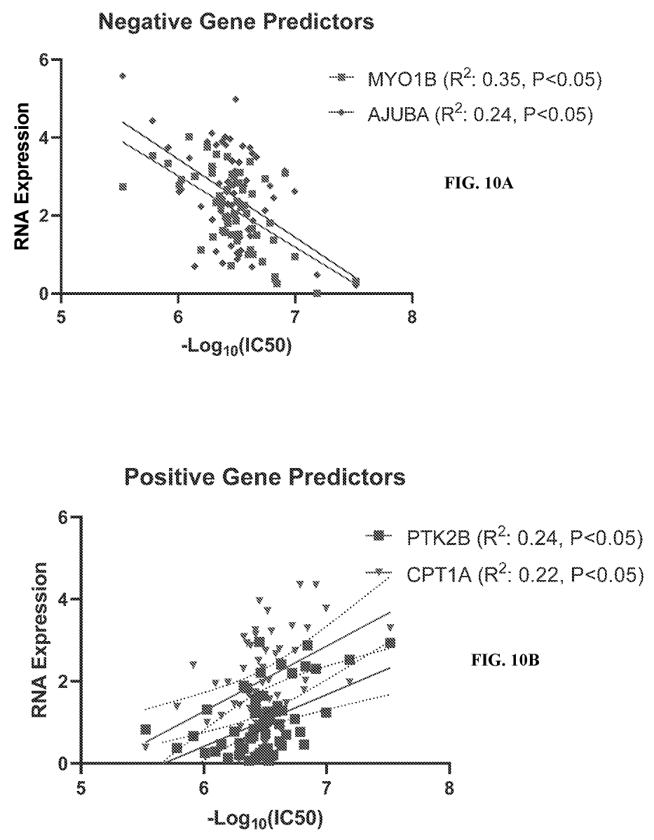

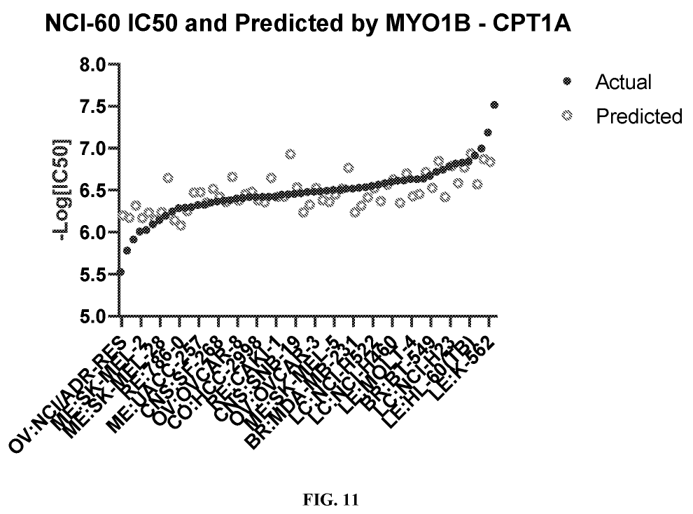

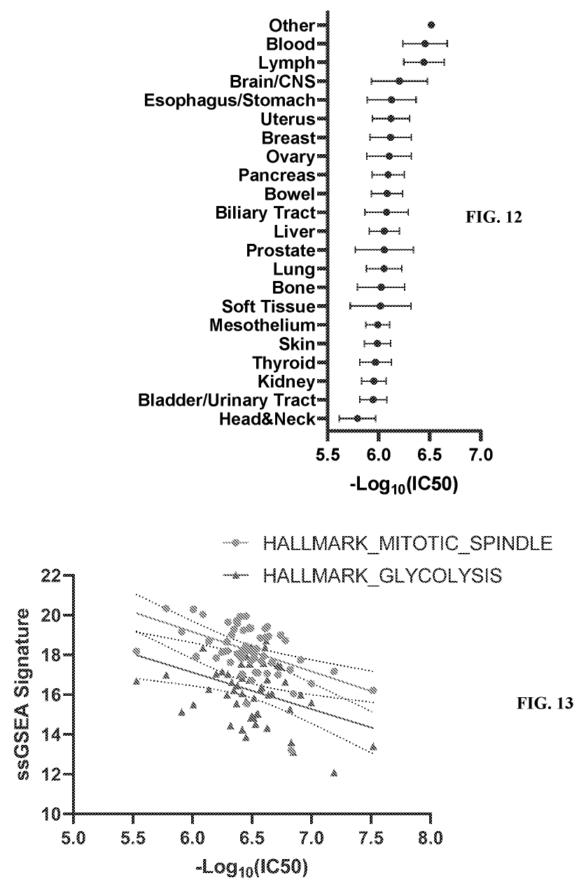

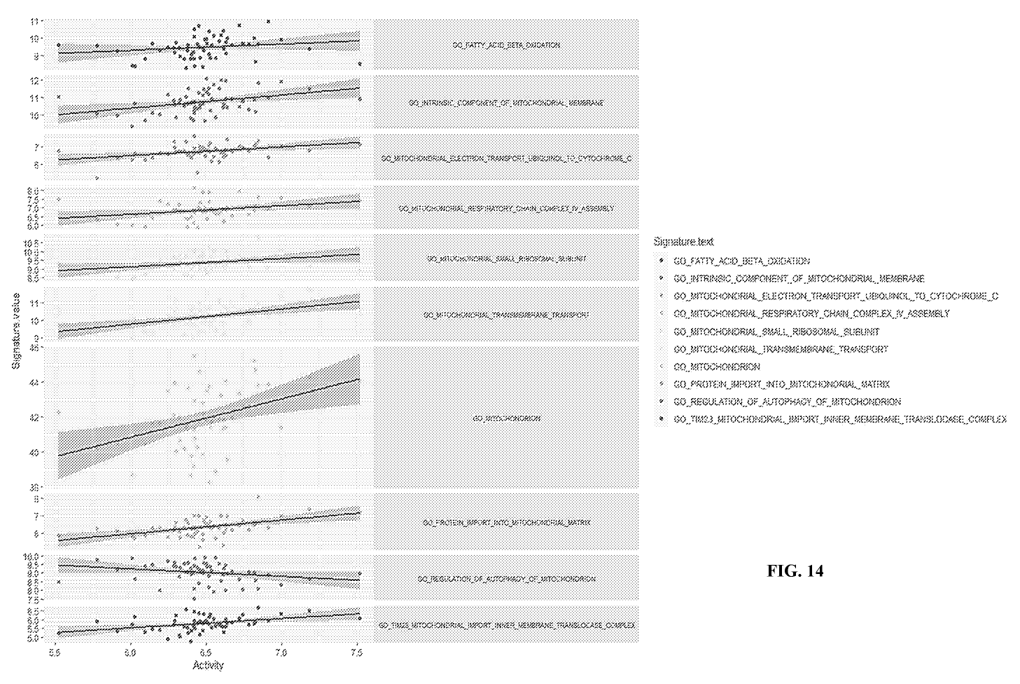

Analysis of the gene expression correlation factors identified a plurality of genes to correlate with drug response (FIG. 9). FIG. 10A shows an example with two genes having negative correlations and FIG. 10B is an example of two genes that have positive correlation. In certain embodiments, the negative gene predictor includes the gene MYO1B and the positive gene predictor includes the CPT1A gene. In certain embodiments, FIG. 11 shows the model of predicted efficacy (as measured by IC₅₀) based on the correlation obtained from the MYO1B and CPT1A genes and the measured experimental IC₅₀ values for JAC-B2 based on NCI-60 data for JAC-B2. In various embodiments, the prediction model can provide tissue-specific predictions on the efficacy of compounds of Formula I or Formula II in the treatment of particular cancers. In certain embodiments, application of the prediction model described herein on gene expression data available from the CCLE (Broad Institute Cancer Cell Line Encyclopedia) shows the efficacy predictions (as measured by -log (IC₅₀)) on the various cancer tissues shown in FIG. 12.

In various embodiments, the gene is at least one gene selected from the group of consisting of the following list of genes in Table 1A, which was compiled from the list of all significant correlating genes by using a threshold of |Estimated Activity|>0.172.

TABLE 1A

Genes With Correlation to Efficacy of Compounds of Formula I or Formula II in Cancer

| Gene Name | Slope | Spearman Coefficient | Spearman p-Value | Pearson Coefficient | Pearson p-Value | Estimated Activity |
|---|---|---|---|---|---|---|
| AKT3 | -0.129068 | -0.409695 | 0.001272 | -0.452925 | 0.000316 | -0.288209 |
| MYO1B | -0.190282 | -0.529443 | 0.000016 | -0.590378 | 0.000001 | -0.276290 |
| LINC00839 | -0.130878 | -0.296283 | 0.022696 | -0.475089 | 0.000143 | -0.267645 |
| ADAMTS1 | -0.092894 | -0.360610 | 0.005021 | -0.414709 | 0.001092 | -0.250441 |
| AFAP1L1 | -0.132525 | -0.408838 | 0.001305 | -0.400373 | 0.001677 | -0.249544 |
| AJUBA | -0.119240 | -0.388468 | 0.002362 | -0.489139 | 0.000085 | -0.245635 |
| HOXA4 | -0.167388 | -0.462932 | 0.000223 | -0.481823 | 0.000112 | -0.245055 |
| RGS20 | -0.107720 | -0.382087 | 0.002824 | -0.420128 | 0.000924 | -0.243016 |
| ST5 | -0.162623 | -0.399778 | 0.001707 | -0.486707 | 0.000093 | -0.241495 |
| UGCG | -0.118158 | -0.417750 | 0.000994 | -0.488093 | 0.000088 | -0.238443 |
| RBMS3 | -0.108180 | -0.320210 | 0.013420 | -0.364797 | 0.004501 | -0.236591 |
| KIRREL | -0.088829 | -0.438054 | 0.000521 | -0.443550 | 0.000434 | -0.236107 |
| PTTG1IP | -0.143777 | -0.334931 | 0.009515 | -0.423738 | 0.000825 | -0.235938 |
| LGALS3BP | -0.076232 | -0.338900 | 0.008649 | -0.518581 | 0.000026 | -0.235327 |
| C15orf52 | -0.115992 | -0.352285 | 0.006212 | -0.422332 | 0.000862 | -0.234536 |
| DLC1 | -0.094408 | -0.427832 | 0.000725 | -0.433621 | 0.000602 | -0.233187 |
| PTPRM | -0.093908 | -0.430060 | 0.000675 | -0.394258 | 0.002003 | -0.231482 |
| IQCJ-SCHIP1 | -0.160202 | -0.421189 | 0.000894 | -0.452935 | 0.000316 | -0.231332 |
| PPP1R15A | -0.162559 | -0.419410 | 0.000945 | -0.492860 | 0.000073 | -0.231321 |
| AMOTL2 | -0.101581 | -0.411841 | 0.001192 | -0.495370 | 0.000066 | -0.229675 |
| UXS1 | -0.170544 | -0.416955 | 0.001019 | -0.442542 | 0.000449 | -0.228700 |
| DAPK1 | -0.113387 | -0.458710 | 0.000258 | -0.405804 | 0.001429 | -0.226887 |
| RIN1 | -0.128515 | -0.378912 | 0.003082 | -0.424642 | 0.000802 | -0.225672 |
| OR2A20P | -0.268895 | -0.392208 | 0.002124 | -0.446649 | 0.000391 | -0.225603 |
| SLC39A13 | -0.162438 | -0.288052 | 0.026942 | -0.448313 | 0.000370 | -0.220916 |
| TMEM2 | -0.168521 | -0.363190 | 0.004695 | -0.461396 | 0.000235 | -0.219415 |
| LRP12 | -0.157518 | -0.352840 | 0.006125 | -0.432311 | 0.000628 | -0.218319 |
| EXT1 | -0.112842 | -0.381449 | 0.002874 | -0.504781 | 0.000046 | -0.217898 |
| WWTR1 | -0.076680 | -0.391625 | 0.002160 | -0.386950 | 0.002466 | -0.217463 |
| PRKCDBP | -0.053838 | -0.266451 | 0.041360 | -0.333704 | 0.009798 | -0.216646 |
| CYTH3 | -0.155590 | -0.419439 | 0.000944 | -0.501364 | 0.000052 | -0.216271 |
| PFKP | -0.175045 | -0.484402 | 0.000101 | -0.548431 | 0.000007 | -0.214780 |
| LPIN3 | -0.136651 | -0.324107 | 0.012271 | -0.346222 | 0.007229 | -0.214679 |
| MOXD1 | -0.094608 | -0.428396 | 0.000712 | -0.402804 | 0.001562 | -0.214477 |
| CTHRC1 | -0.082327 | -0.315897 | 0.014797 | -0.408732 | 0.001309 | -0.214049 |
| LINC01117 | -0.329477 | -0.401032 | 0.001645 | -0.421348 | 0.000889 | -0.213171 |
| CCDC93 | -0.301970 | -0.372892 | 0.003630 | -0.495859 | 0.000065 | -0.212889 |
| APBB1 | -0.091852 | -0.310102 | 0.016838 | -0.388734 | 0.02345 | -0.211628 |
| TMEM65 | -0.333706 | -0.283585 | 0.029514 | -0.486171 | 0.000095 | -0.211569 |
| PIK3 C2A | -0.213046 | -0.333031 | 0.009956 | -0.426305 | 0.000761 | -0.211129 |
| FRMD6 | -0.100770 | -0.398288 | 0.001783 | -0.448169 | 0.000371 | -0.209601 |
| ETS1 | -0.103369 | -0.362133 | 0.004826 | -0.421476 | 0.000886 | -0.209322 |
| MMP14 | -0.055965 | -0.316491 | 0.014601 | -0.346591 | 0.007163 | -0.209031 |
| SH2D5 | -0.206737 | -0.420556 | 0.000912 | -0.469529 | 0.000176 | -0.208597 |
| PARVA | -0.142567 | -0.419942 | 0.000929 | -0.475150 | 0.000143 | -0.208433 |
| SPARC | -0.033547 | -0.250387 | 0.055786 | -0.341591 | 0.008101 | -0.208228 |
| SPTBN1 | -0.173135 | -0.444571 | 0.000419 | -0.477626 | 0.000131 | -0.207589 |
| RBMS2 | -0.163552 | -0.325229 | 0.011957 | -0.459121 | 0.000255 | -0.207220 |
| NNMT | -0.043697 | -0.235889 | 0.072082 | -0.338699 | 0.008691 | -0.207213 |
| ENO2 | -0.103936 | -0.328760 | 0.011012 | -0.412926 | 0.001153 | -0.206834 |
| ASPHD1 | -0.109897 | -0.319072 | 0.013772 | -0.368450 | 0.004088 | -0.206716 |
| PLCB4 | -0.138774 | -0.572798 | 0.000002 | -0.557467 | 0.000005 | -0.206496 |
| ASPH | -0.095921 | -0.306901 | 0.018065 | -0.459324 | 0.000253 | -0.206422 |
| ANXA5 | -0.118629 | -0.400064 | 0.001693 | -0.460646 | 0.000241 | -0.205940 |
| PPP2R3A | -0.166646 | -0.326042 | 0.011733 | -0.412703 | 0.001161 | -0.203641 |
| SYDE1 | -0.082292 | -0.257138 | 0.049293 | -0.336612 | 0.009139 | -0.203591 |
| AASS | -0.166045 | -0.307400 | 0.017869 | -0.331444 | 0.010338 | -0.203239 |
| PYROXD2 | -0.272676 | -0.369756 | 0.003948 | -0.439686 | 0.000493 | -0.202053 |
| OR2A9P | -0.321594 | -0.425563 | 0.000779 | -0.399908 | 0.001700 | -0.201961 |
| LMCD1 | -0.100492 | -0.416295 | 0.001040 | -0.426472 | 0.000757 | -0.201085 |
| TLDC1 | -0.160586 | -0.327094 | 0.011449 | -0.385649 | 0.002557 | -0.201054 |
| ZC2HC1A | -0.322078 | -0.288436 | 0.026730 | -0.407701 | 0.001350 | -0.200655 |
| DCBLD2 | -0.067310 | -0.295387 | 0.023128 | -0.362331 | 0.004801 | -0.200515 |
| ABLIM3 | -0.086784 | -0.397896 | 0.001803 | -0.364833 | 0.004497 | -0.200470 |
| RAB34 | -0.045209 | -0.284007 | 0.029263 | -0.325816 | 0.011795 | -0.200364 |
| CYR61 | -0.042335 | -0.311055 | 0.016487 | -0.353986 | 0.005950 | -0.199993 |
| TGFB1I1 | -0.067749 | -0.256287 | 0.050076 | -0.326970 | 0.011482 | -0.199048 |
| FAM84B | -0.093311 | -0.332734 | 0.010026 | -0.385468 | 0.002570 | -0.198473 |
| FHL2 | -0.087455 | -0.385131 | 0.002595 | -0.487196 | 0.000091 | -0.198087 |
| KLHL29 | -0.203682 | -0.373121 | 0.003608 | -0.393416 | 0.002052 | -0.197979 |
| CC2D2A | -0.222654 | -0.263742 | 0.043551 | -0.404456 | 0.001487 | -0.197939 |
| CTTNBP2NL | -0.221995 | -0.335603 | 0.009363 | -0.486083 | 0.000095 | -0.197797 |
| COL4A2 | -0.046960 | -0.324410 | 0.012185 | -0.320111 | 0.013450 | -0.196809 |
| DZIP1L | -0.218092 | -0.395716 | 0.001921 | -0.402316 | 0.001584 | -0.195410 |
| P3H2 | -0.058844 | -0.292580 | 0.024530 | -0.323276 | 0.012509 | -0.195068 |
| FARP1 | -0.148352 | -0.400999 | 0.001647 | -0.475833 | 0.000140 | -0.194934 |
| DKK3 | -0.052929 | -0.319323 | 0.013694 | -0.330173 | 0.010652 | -0.194301 |
| TTL | -0.157769 | -0.334838 | 0.009536 | -0.433621 | 0.000602 | -0.193582 |
| MAP4K3 | -0.206917 | -0.268214 | 0.039985 | -0.376794 | 0.003266 | -0.193261 |
| FAM160A1 | -0.223936 | -0.354263 | 0.005908 | -0.357285 | 0.005470 | -0.193032 |
| CD40 | -0.083494 | -0.336687 | 0.009123 | -0.361308 | 0.004931 | -0.192787 |
| MYH9 | -0.105170 | -0.313539 | 0.015601 | -0.366096 | 0.004350 | -0.192777 |
| RIN2 | -0.103555 | -0.307490 | 0.017834 | -0.337564 | 0.008932 | -0.192509 |
| KCNIP3 | -0.119392 | -0.332130 | 0.010171 | -0.395761 | 0.001918 | -0.191743 |
| FOSL1 | -0.056920 | -0.371221 | 0.003796 | -0.399408 | 0.001726 | -0.191535 |
| ABTB2 | -0.188199 | -0.363628 | 0.004641 | -0.419271 | 0.000949 | -0.191399 |
| PTPRG | -0.116445 | -0.388667 | 0.002349 | -0.346943 | 0.007101 | -0.191320 |
| MYO1E | -0.143991 | -0.424443 | 0.000807 | -0.447974 | 0.000374 | -0.191220 |
| MCAM | -0.047818 | -0.282884 | 0.029936 | -0.322997 | 0.012589 | -0.191178 |
| MICAL3 | -0.141780 | -0.368825 | 0.004047 | -0.407575 | 0.001355 | -0.190552 |
| DNAJC13 | -0.246824 | -0.259359 | 0.047297 | -0.444317 | 0.000423 | -0.190548 |
| MOCS1 | -0.145537 | -0.256420 | 0.049953 | -0.330527 | 0.010564 | -0.190362 |
| NEK6 | -0.152732 | -0.391180 | 0.002188 | -0.487060 | 0.000092 | -0.189387 |
| BIN1 | -0.105852 | -0.319881 | 0.013521 | -0.391875 | 0.002145 | -0.189368 |
| SLC35F5 | -0.280434 | -0.439025 | 0.000504 | -0.520688 | 0.000024 | -0.189293 |
| HKDC1 | -0.103583 | -0.402043 | 0.001597 | -0.373007 | 0.003619 | -0.189039 |
| ITGA3 | -0.060853 | -0.323237 | 0.012520 | -0.396947 | 0.001854 | -0.188828 |
| LBX2-AS1 | -0.164607 | -0.436337 | 0.000551 | -0.411223 | 0.001214 | -0.188640 |
| RALB | -0.155564 | -0.322565 | 0.012715 | -0.422601 | 0.000855 | -0.188388 |
| EPB41L2 | -0.130958 | -0.393022 | 0.002076 | -0.438310 | 0.000516 | -0.188318 |
| FRMD4A | -0.093133 | -0.371250 | 0.003793 | -0.334349 | 0.009648 | -0.188315 |
| ARHGEF5 | -0.110164 | -0.348890 | 0.006765 | -0.351252 | 0.006376 | -0.188271 |
| HOXA3 | -0.132408 | -0.406084 | 0.001417 | -0.416007 | 0.001049 | -0.188151 |
| PRKCZ | -0.106613 | -0.310592 | 0.016657 | -0.336754 | 0.009108 | -0.187851 |
| EPS8L2 | -0.058312 | -0.274317 | 0.035509 | -0.319338 | 0.013689 | -0.18-532 |
| PPFEBP1 | -0.144554 | -0.313218 | 0.015713 | -0.431730 | 0.000640 | -0.187197 |
| MAP3K9 | -0.201641 | -0.363984 | 0.004598 | -0.389456 | 0.002297 | -0.187123 |
| BTBD10 | -0.148339 | -0.315347 | 0.014982 | -0.366733 | 0.004278 | -0.187056 |
| PLEKHG3 | -0.162909 | -0.358071 | 0.005361 | -0.404200 | 0.001499 | -0.187020 |
| SAMD4A | -0.154939 | -0.346007 | 0.007268 | -0.390158 | 0.002252 | -0.186857 |
| ELK3 | -0.101152 | -0.287589 | 0.027200 | -0.340586 | 0.008302 | -0.186828 |
| ACVR1 | -0.130043 | -0.274083 | 0.035673 | -0.339634 | 0.008496 | -0.186741 |
| APAF1 | -0.362046 | -0.301679 | 0.020230 | -0.461988 | 0.000230 | -0.186454 |
| MPRIP | -0.161048 | -0.343225 | 0.007783 | -0.421092 | 0.000896 | -0.186332 |
| ALS2 | -0.274821 | -0.304547 | 0.019015 | -0.439515 | 0.000496 | -0.186054 |
| IL32 | -0.042939 | -0.249273 | 0.056923 | -0.346194 | 0.007234 | -0.185839 |
| TRIP12 | -0.194966 | -0.266135 | 0.041611 | -0.381418 | 0.002877 | -0.185802 |
| TM4SF1 | -0.050127 | -0.258896 | 0.047707 | -0.372188 | 0.003699 | -0.185669 |
| SH3BP4 | -0.117594 | -0.447990 | 0.000374 | -0.467655 | 0.000188 | -0.185329 |
| DENND3 | -0.182301 | -0.277423 | 0.033397 | -0.395460 | 0.001935 | -0.184670 |
| KIF3C | -0.136787 | -0.323621 | 0.012410 | -0.376817 | 0.003264 | -0.183978 |
| LINC01116 | -0.080777 | -0.304611 | 0.018989 | -0.326878 | 0.011507 | -0.183930 |
| COL27A1 | -0.124780 | -0.393781 | 0.002031 | -0.380464 | 0.002953 | -0.183551 |
| CYFIP1 | -0.152222 | -0.284353 | 0.029058 | -0.397638 | 0.001817 | -0.183275 |
| CLASP1 | -0.212189 | -0.301996 | 0.020093 | -0.343259 | 0.007777 | -0.183119 |
| FZD6 | -0.150582 | -0.236418 | 0.071426 | -0.495583 | 0.000066 | -0.183107 |
| CAPG | -0.053788 | -0.327883 | 0.011240 | -0.360992 | 0.004971 | -0.182932 |
| RASAL2 | -0.185518 | -0.322482 | 0.012739 | -0.401607 | 0.001618 | -0.182921 |
| USP43 | -0.282309 | -0.289948 | 0.025909 | -0.351431 | 0.006347 | -0.182654 |
| MATN2 | -0.094735 | -0.322185 | 0.012827 | -0.413815 | 0.001122 | -0.182554 |
| ARHGAP5 | -0.159884 | -0.271428 | 0.037573 | -0.359760 | 0.005132 | -0.182268 |
| TGM2 | -0.035800 | -0.304071 | 0.019213 | -0.293020 | 0.024306 | -0.182149 |
| DFNA5 | -0.102234 | -0.277675 | 0.033230 | -0.400090 | 0.001691 | -0.181976 |
| PVR | -0.126847 | -0.411958 | 0.001187 | -0.445297 | 0.000409 | -0.181265 |
| PHLDB1 | -0.102547 | -0.320285 | 0.013397 | -0.378645 | 0.003105 | -0.181201 |
| HOXD4 | -0.214374 | -0.455155 | 0.000292 | -0.440121 | 0.000486 | -0.180931 |
| SRGAP1 | -0.123480 | -0.370467 | 0.003874 | -0.393613 | 0.002041 | -0.180898 |
| PACSIN2 | -0.167460 | -0.351846 | 0.006281 | -0.402913 | 0.001557 | -0.180689 |
| MAMLD1 | -0.086848 | -0.294306 | 0.023660 | -0.307682 | 0.017759 | -0.180557 |
| BOK | -0.115306 | -0.319910 | 0.013512 | -0.430734 | 0.000661 | -0.180224 |
| PTPN9 | -0.184792 | -0.330309 | 0.010618 | -0.374987 | 0.003430 | -0.179803 |
| C3orf52 | -0.194364 | -0.339256 | 0.008574 | -0.376831 | 0.003263 | -0.179787 |
| ITGB1 | -0.088210 | -0.184398 | 0.162082 | -0.384440 | 0.002645 | -0.179684 |
| CARD10 | -0.108126 | -0.232557 | 0.076315 | -0.348064 | 0.006906 | -0.179597 |
| HIF1A | -0.103724 | -0.226187 | 0.084960 | -0.406244 | 0.001410 | -0.179443 |
| MGAT4B | -0.131519 | -0.299478 | 0.021207 | -0.394870 | 0.001968 | -0.179391 |
| TMEM51 | -0.158363 | -0.484877 | 0.000099 | -0.509500 | 0.000038 | -0.179108 |
| DOCK7 | -0.187222 | -0.264060 | 0.043289 | -0.358028 | 0.005366 | -0.178797 |
| EPB41L3 | -0.083192 | -0.193408 | 0.142185 | -0.343053 | 0.007816 | -0.178779 |
| MICALL1 | -0.135550 | -0.296360 | 0.022659 | -0.356371 | 0.005599 | -0.178248 |
| RHPN2 | -0.143967 | -0.379146 | 0.003062 | -0.390001 | 0.002262 | -0.178087 |
| CRIM1 | -0.068918 | -0.253394 | 0.052814 | -0.321959 | 0.012893 | -0.178014 |
| LPP | -0.166596 | -0.308805 | 0.017326 | -0.416431 | 0.001036 | -0.177924 |
| EFNA5 | -0.143096 | -0.394746 | 0.001975 | -0.433888 | 0.000597 | -0.177868 |
| FAM50B | -0.107167 | -0.190330 | 0.148765 | -0.300816 | 0.020609 | -0.177791 |
| RHBDF1 | -0.122164 | -0.279552 | 0.032010 | -0.368148 | 0.004120 | -0.177504 |
| L1CAM | -0.048243 | -0.220199 | 0.093771 | -0.291215 | 0.025238 | -0.177245 |
| CALD1 | -0.050316 | -0.222800 | 0.089860 | -0.313130 | 0.015744 | -0.177163 |
| AXL | -0.031425 | -0.213738 | 0.104063 | -0.271414 | 0.037583 | -0.177049 |
| GLS | -0.109745 | -0.442964 | 0.000443 | -0.418080 | 0.000984 | -0.177019 |
| GPRC5B | -0.070072 | -0.357342 | 0.005462 | -0.313865 | 0.015487 | -0.176442 |
| TPM1 | -0.056922 | -0.319029 | 0.013786 | -0.359076 | 0.005224 | -0.176116 |
| DOK4 | -0.129210 | -0.243202 | 0.063443 | -0.411477 | 0.001205 | -0.176113 |
| TRNP1 | -0.080301 | -0.361227 | 0.004941 | -0.336133 | 0.009245 | -0.175778 |
| MTCL1 | -0.104643 | -0.345534 | 0.007353 | -0.365364 | 0.004435 | -0.175695 |
| ARMC9 | -0.146191 | -0.356235 | 0.005619 | -0.425700 | 0.000776 | -0.175429 |
| FSTL3 | -0.068808 | -0.278496 | 0.032691 | -0.333946 | 0.009741 | -0.175253 |
| ITGAV | -0.127501 | -0.297491 | 0.022123 | -0.395839 | 0.001914 | -0.175186 |
| ASAP1 | -0.103790 | -0.302518 | 0.019869 | -0.381968 | 0.002833 | -0.174990 |
| DYRK2 | -0.189959 | -0.271545 | 0.037487 | -0.330053 | 0.010682 | -0.174763 |
| ACTN1 | -0.107402 | -0.382443 | 0.002796 | -0.495915 | 0.000065 | -0.174743 |
| C7orf31 | -0.337021 | -0.340566 | 0.008306 | -0.380632 | 0.002940 | -0.174577 |
| CHST3 | -0.099738 | -0.286570 | 0.027774 | -0.328784 | 0.011006 | -0.174143 |
| RGS12 | -0.208888 | -0.357667 | 0.005417 | -0.441074 | 0.000471 | -0.173795 |
| EPDR1 | -0.065179 | -0.369419 | 0.003983 | -0.349602 | 0.006645 | -0.173768 |
| EGFR | -0.052350 | -0.265145 | 0.042405 | -0.293575 | 0.024025 | -0.173749 |
| LUZP1 | -0.182932 | -0.311314 | 0.016393 | -0.370584 | 0.003862 | -0.173603 |
| RIPK4 | -0.082985 | -0.358222 | 0.005340 | -0.310017 | 0.016870 | -0.173438 |
| RAI14 | -0.085005 | -0.247578 | 0.058685 | -0.354027 | 0.005944 | -0.173411 |
| ITPR3 | -0.093125 | -0.397697 | 0.001814 | -0.343640 | 0.007704 | -0.173398 |
| PTPRJ | -0.136096 | -0.388112 | 0.002386 | -0.387891 | 0.002401 | -0.173387 |
| PKN2 | -0.183339 | -0.198866 | 0.131053 | -0.341817 | 0.008056 | -0.173072 |
| SIRPA | -0.050880 | -0.333699 | 0.009799 | -0.301708 | 0.020218 | -0.173041 |
| CCNJL | -0.159949 | -0.393115 | 0.002070 | -0.418474 | 0.000972 | -0.172745 |
| SPATA20 | -0.098367 | -0.322014 | 0.012877 | -0.422620 | 0.000855 | -0.172732 |
| ASXL1 | -0.203621 | -0.212920 | 0.105426 | -0.364068 | 0.004588 | -0.172670 |
| PRRG1 | -0.183621 | -0.310280 | 0.016772 | -0.379048 | 0.003071 | -0.172604 |
| RINL | 0.390850 | 0.390599 | 0.002224 | 0.353161 | 0.006076 | 0.172756 |
| HIST3H2BB | 0.090033 | 0.252093 | 0.054083 | 0.347566 | 0.006992 | 0.173584 |
| HIST1H1E | 0.128717 | 0.294602 | 0.023513 | 0.397485 | 0.001825 | 0.176343 |
| HIST1H2AD | 0.056434 | 0.332739 | 0.010025 | 0.348358 | 0.006855 | 0.177483 |
| MDH2 | 0.143646 | 0.448604 | 0.000366 | 0.349174 | 0.006717 | 0.181138 |
| SLC22A23 | 0.193276 | 0.426582 | 0.000754 | 0.423267 | 0.000837 | 0.183032 |
| ALDOC | 0.069486 | 0.303774 | 0.019337 | 0.333552 | 0.009833 | 0.185943 |
| GEMIN8 | 0.250656 | 0.513508 | 0.000032 | 0.435252 | 0.000571 | 0.185987 |
| PTK2B | 0.187689 | 0.384371 | 0.002650 | 0.486707 | 0.000093 | 0.195572 |
| KRTCAP2 | 0.179463 | 0.440275 | 0.000484 | 0.448406 | 0.000368 | 0.199203 |
| JTB | 0.155931 | 0.486448 | 0.000094 | 0.397098 | 0.001846 | 0.203022 |
| CPT1A | 0.135656 | 0.450240 | 0.000346 | 0.464544 | 0.000210 | 0.208368 |
| S100A4 | 0.043479 | 0.407043 | 0.001377 | 0.391547 | 0.002165 | 0.218960 |

Analysis of the GSEA (http://www.gsea-msigdb.org) hallmark pathways set identified 16 signatures to be significantly correlated (based on either Pearson’s or Spearman’s correlation test). Linear regression was employed to extract slopes and estimate the change in -log(IC₅₀) over the inter-quartile range (IQR). In various embodiments, the significant hallmark signatures, rated by their -log(IC₅₀) change over the IQR are the following list of signatures in Table 1B which was compiled from the list of all significant correlating genes by using a threshold of |Estimated Activity|1>0.1:

TABLE 1B

Significant Hallmark gene signatures with Correlation to Efficacy of Compounds of Formula I or Formula II in Cancer

| Signature Name (Hallmark) | Slope | Spearman Coeff. | Spearman p-Value | Pearson Coeff. | Pearson p-Value | Estimated Activity |
|---|---|---|---|---|---|---|
| MITOTIC_SPINDLE | 0.118 | -0.467 | 0 | -0.483 | 0 | -0.189 |
| EPITHELIAL_MESENCHYMAL_TRANSITION | 0.016 | -0.232 | 0.077 | -0.322 | 0.013 | -0.18 |
| GLYCOLYSIS | 0.078 | -0.281 | 0.031 | -0.379 | 0.003 | -0.177 |
| UV RESPONSE DN | -0.04 | -0.288 | 0.027 | -0.385 | 0.003 | -0.176 |
| HEDGEHOG_SIGNALING | 0.077 | -0.314 | 0.015 | -0.35 | 0.007 | -0.162 |
| APOPTOSIS | 0.077 | -0.194 | 0.141 | -0.333 | 0.01 | -0.141 |
| COAGULATION | 0.047 | -0.279 | 0.032 | -0.352 | 0.006 | -0.131 |
| COMPLEMENT | 0.059 | -0.281 | 0.031 | -0.326 | 0.012 | -0.13 |
| TGF_BETA_SIGNALING | 0.071 | -0.159 | 0.228 | -0.272 | 0.037 | -0.125 |
| TNFA_SIGNALING_VIA_NFKB | 0.028 | -0.221 | 0.093 | -0.292 | 0.025 | -0.125 |
| HYPOXIA | -0.04 | -0.204 | 0.121 | -0.3 | 0.021 | -0.122 |
| APICAL_JUNCTION | 0.039 | -0.284 | 0.029 | -0.311 | 0.016 | -0.12 |
| APICAL_SURFACE | 0.079 | -0.31 | 0.017 | -0.278 | 0.033 | -0.117 |
| ANDROGEN_RESPONSE | 0.088 | -0.257 | 0.049 | -0.254 | 0.052 | -0.103 |
| MYOGENESIS | 0.031 | -0.188 | 0.155 | -0.268 | 0.04 | -0.1 |
| IL2_STAT5_SIGNALING | 0.058 | -0.316 | 0.015 | -0.263 | 0.044 | -0.088 |

Analysis of genetic pathway signatures also revealed mitochondrial pathways to be strongly predictive of response. In various embodiments, the significant hallmark signatures, rated by their -log(IC₅₀) change over the IQR are the following list of signatures in Table 1C which was compiled from the list of all significant correlating genes by using a threshold of |Estimated Activity|>0.1:

TABLE 1C

Significant Mitochondrial gene signatures with Correlation to Efficacy of Compounds of Formula I or Formula II in Cancer

| Signature Name | Slope | Spearman Coeff. | Spearman p-Value | Pearson Coeff. | Pearson p-Value | Estimated Activity |
|---|---|---|---|---|---|---|
| MITOCHONDRION | 0.072 | 0.398 | 0.002 | 0.399 | 0.002 | 0.196 |
| PROTEIN_IMPORT_INTO_MITOCHONDRIAL_MATRIX | 0.296 | 0.449 | 0 | 0.482 | 0 | 0.179 |
| MITOCHONDRIAL_TRANSMEMBRANE_TRANSPORT | 0.252 | 0.474 | 0 | 0.465 | 0 | 0.179 |
| INTRINSIC_COMPONENT_OFMITOCHONDRIAL_INNER_MEMBRANE | 0.197 | 0.316 | 0.015 | 0.334 | 0.01 | 0.178 |
| MITOCHONDRION_ORGANIZATION | 0.093 | 0.318 | 0.014 | 0.294 | 0.024 | 0.157 |
| INTRINSIC_COMPONENT_OF_MITOCHONDRIAL_MEMBRANE | 0.171 | 0.386 | 0.003 | 0.359 | 0.005 | 0.157 |
| INNER_MITOCHONDRIAL_MEMBRANE_PROTEIN_COMPLEX | 0.123 | 0.232 | 0.077 | 0.301 | 0.021 | 0.148 |
| TIM23_MITOCHONDRIAL_IMPORT_INNER_MEMBRANE_TRANSLOCASE_COMPLEX | 0.29 | 0.412 | 0.001 | 0.393 | 0.002 | 0.147 |
| MITOCHONDRIAL_ELECTRON_TRANSPORTUBIQUINOLTO_CYTOCHROME_C | 0.289 | 0.358 | 0.005 | 0.378 | 0.003 | 0.131 |
| MITOCHONDRION | 0.073 | 0.301 | 0.02 | 0.267 | 0.041 | 0.129 |
| NEGATIVE_REGULATION_OF_RELEASE_OF_CYTOCHROME_C_FROM_MITOCHONDRIA | 0.169 | 0.295 | 0.023 | 0.289 | 0.027 | 0.128 |
| MITOCHONDRIAL_SMALL_RIBOSOMAL_SUBUNIT | 0.189 | 0.324 | 0.012 | 0.298 | 0.022 | 0.126 |
| MITOCHONDRIAL_TRANSLATIONAL_TERMINATION | 0.081 | 0.299 | 0.021 | 0.261 | 0.046 | 0.117 |
| MITOCHONDRIAL_RESPIRATORY_CHAIN_COMPLEX_ASSEMBLY | 0.094 | 0.277 | 0.034 | 0.233 | 0.076 | 0.114 |
| MITOCHONDRIAL_RNA_PROCESSING | 0.182 | 0.308 | 0.018 | 0.256 | 0.05 | 0.107 |
| MITOCHONDRIAL_RESPIRATORY_CHAIN_COMPLEX_IV_ASSEMBLY | 0.188 | 0.341 | 0.008 | 0.304 | 0.019 | 0.097 |

### Combination Therapies

The compounds useful within the methods described herein can be used in combination with one or more additional therapeutic agents useful for treating cancer. These additional therapeutic agents may comprise compounds that are commercially available or synthetically accessible to those skilled in the art. These additional therapeutic agents are known to treat or reduce the symptoms, of cancer.

In certain embodiments, the compounds described herein can be used in combination with radiation therapy. In other embodiments, the combination of administration of the compounds described herein and application of radiation therapy is more effective in treating or preventing cancer than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat cancer in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 µg to about 10,000 mg, about 20 µg to about 9,500 mg, about 40 µg to about 9,000 mg, about 75 µg to about 8,500 mg, about 150 µg to about 7,500 mg, about 200 µg to about 7,000 mg, about 350 µg to about 6,000 mg, about 500 µg to about 5,000 mg, about 750 µg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α -lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described hrein by pH-independent swelling.

In some embodiments the compound of Formula I or Formula II is present in any of the oral formulations or dosage forms described herein. In various embodiments, the compound of Formula I or Formula II is formulated in an oral solution, syrup, capsule, or tablet containing about 20 to about 100% (w/v) glycerol and about 0 to about 100 mg/mL of a bile acid salt. The capsules can, in some embodiments, be liquid filled capsules. The oral dosage forms of compounds of Formula I or Formula II can be suitable coated by any of the enteric coatings described herein. Suitable bile acid salts include alkali metal salts of cholic, deoxycholic, chenodeoxycholic, and/or lithocholic acids. Suitable alkali metals include lithium, sodium, and potassium. In various embodiments, the bile acid salt is sodium deoxycholate (sodium salt of deoxycholic acid).

In various embodiments, the oral compositions of compounds of Formula I or Formula II described herein contain at least about, greater than, or less than about 0, 5, 10, 15, 20, 25, 30, 35, 40, 45, 50% (w/v) of glycerol. In various embodiments, the oral compositions of compounds of Formula I or Formula II described herein contain at least about, greater than, or less than about 5, 10, 12.5, 15, 20, 25, 30, 30, 35, 40, 45, 50, 55, 60, 65, 70, 75, 80, 85, 90, 95, or 100 mg/mL of sodium deoxycholate. In various embodiments, the oral compositions of compounds of Formula I or Formula II described herein contain glycerol, bile acid salts, water and one or more excipients that do not reduce the solubility of the compound of Formula I or Formula II in the oral dosage form.

### Parenteral Administration

In various embodiments, compounds of Formula I or Formula II can be formulated for parenteral administration, such as IV or IM, in a formulation that contains 0-30% (w/v) glycerol, 0-100 mg/mL of a bile acid salt, and 0-100 mg/mL of serum albumin. Suitable bile acid salts include alkali metal salts of cholic, deoxycholic, chenodeoxycholic, and/or lithocholic acids. Suitable alkali metals include lithium, sodium, and potassium. In various embodiments, the bile acid salt is sodium deoxycholate (sodium salt of deoxycholic acid). Suitable serum albumins include mammalian serum albumin, such as murine, porcine, bovine, and human serum albumin, and the like. In various embodiments, the serum albumin is bovine serum albumin.

In various embodiments, the parenteral compositions of compounds of Formula I or Formula II described herein contain at least about, greater than, or less than about 0, 5, 10, 15, 20, 25, 30, 35, 40, 45, 50% (w/v) of glycerol. In various embodiments, the parenteral compositions of compounds of Formula I or Formula II described herein contain at least about, greater than, or less than about 5, 10, 12.5, 15, 20, 25, 30, 30, 35, 40, 45, 50, 55, 60, 65, 70, 75, 80, 85, 90, 95, or 100 mg/mL of sodium deoxycholate. In various embodiments, the parenteral compositions of compounds of Formula I or Formula II described herein contains at least about, greater than, or less than about 5, 10, 15, 20, 25, 30, 30, 35, 40, 45, 50, 55, 60, 65, 70, 75, 80, 85, 90, 95, or 100 mg/mL of bovine serum albumin (BSA).

In various embodiments, the parenteral compositions of compounds of Formula I or Formula II described herein contain glycerol, serum albumin, bile acid salts, water and one or more excipients that do not reduce the solubility of the compound of Formula I or Formula II in the parenteral dosage form.

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer’s solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Pat. Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In certain embodiments, the compound(s) described herein are administered to a patient, alone or in combination with another pharmaceutical agent, using a sustained release formulation. In certain embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of cancer in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient’s status does improve, upon the doctor’s discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient’s conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### Example 1

NCI-N87 and OE33 cancer cells were grown in normal culture media (DMEM media, 10% FBS, 1% L-glutamine, 1% Penicillin-Streptomycin) at normal conditions (37° C., 5% CO₂). The cells were trypsinized and plated at 1 thousand cells per well and in 96-well plates.

The cells were then treated with CDI (JAC-B2, also known as dithiazanine iodide) for 5 days. Cell viability was assessed using pico-green dsDNA measurement and IC_{50S} were calculated using Graphpad® (GraphPad Software, San Diego, CA, USA) by employing normalized nonlinear regression models.

### Example 2

MIA-PaCa2 pancreatic cancer cells were purchased from the American Type Culture Collection (ATCC, Manassas, VA, USA) and grown in normal culture media (DMEM media, 10% FBS, 1% L-glutamine, 1% Penicillin-Streptomycin) at normal conditions (37° C., 5% CO₂). Cells were trypsinized and plated at 1 thousand cells per well and in 96-well plates. The cells were then treated with a variety of compounds having the Formula I structure (JAC-B1, JAC-B1.5, JAC-B2, JAC-B2.5, JAC-B3, JAC-R1, JAC-R2, JAC-R3 also known as dithiazanine iodide) for 5 days. Cell viability was assessed using pico-green dsDNA measurement and IC_{50S} were calculated using GraphPad® (GraphPad Software, San Diego, CA, USA) by employing normalized nonlinear regression models. A summary of the combined efficacy of a compound of Formula I and an additional therapeutic agent is show in Table 2.

TABLE 2

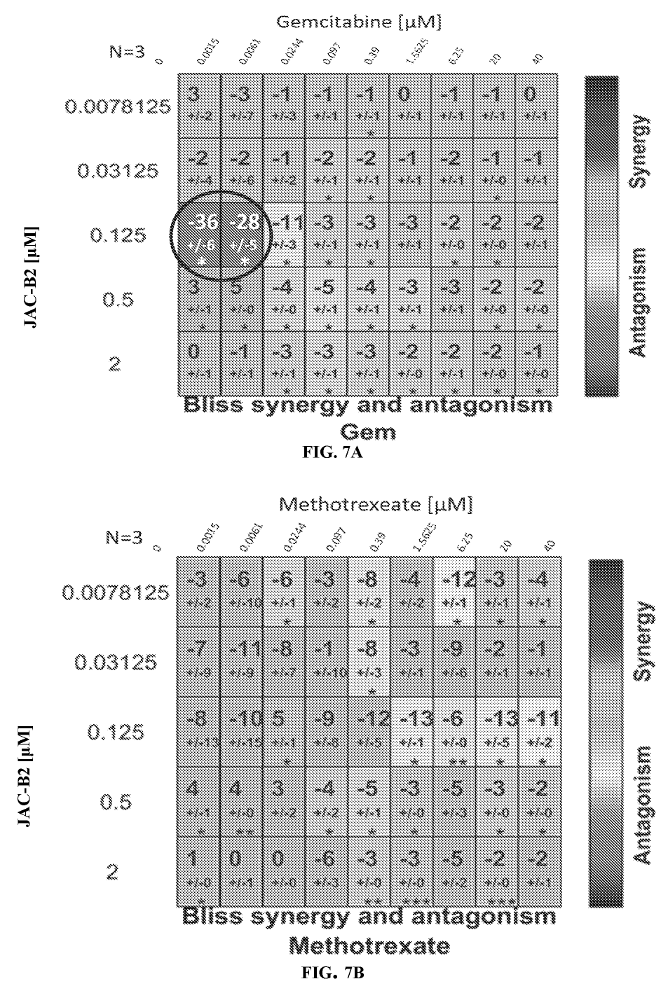

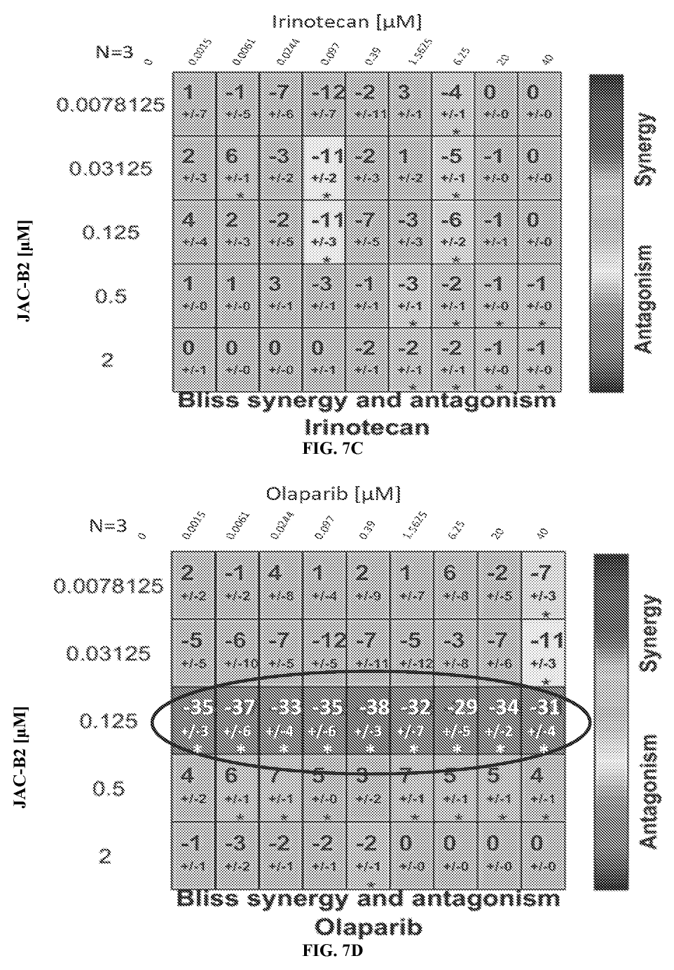

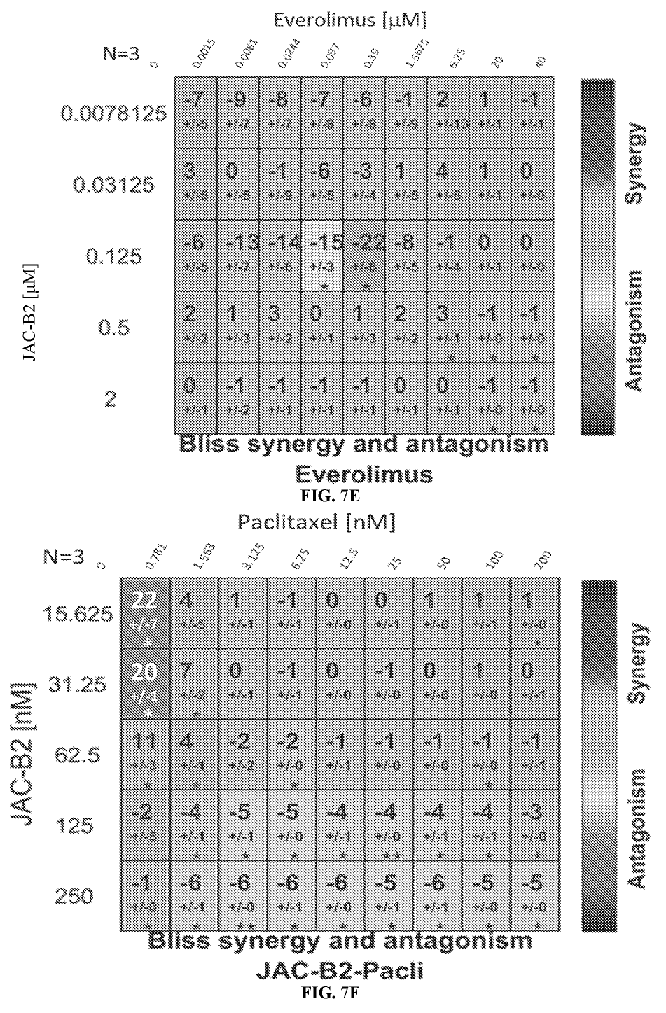

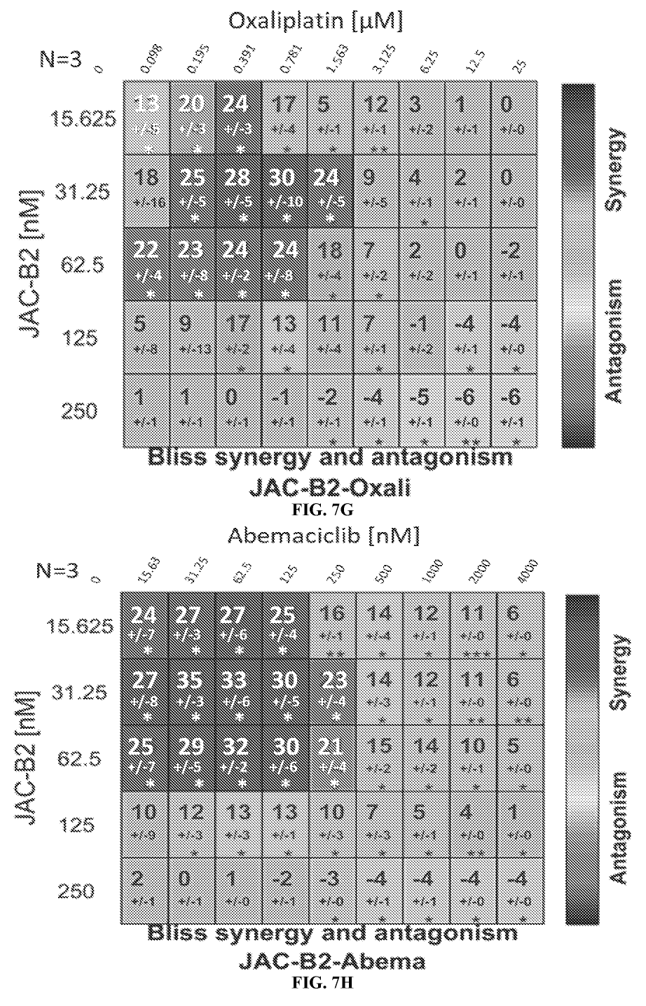

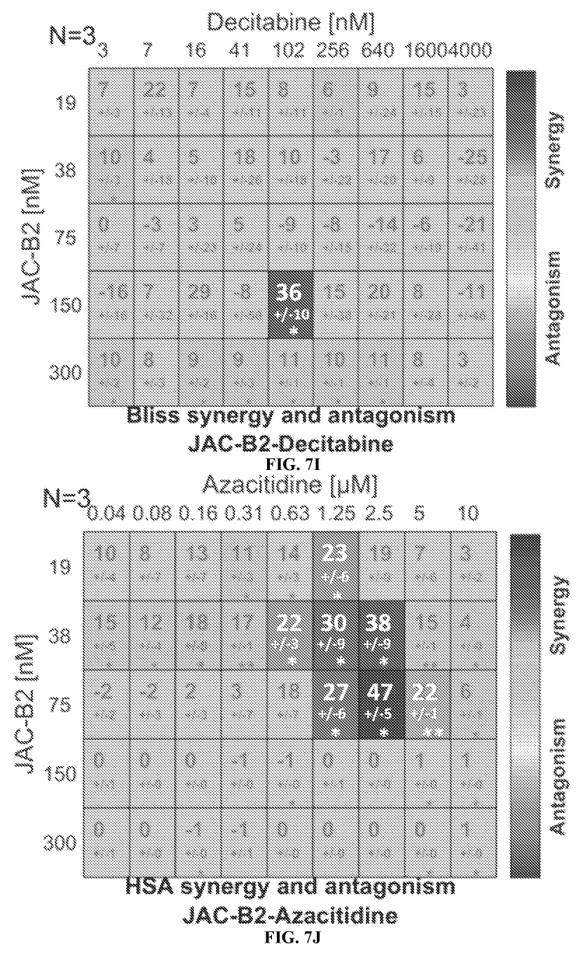

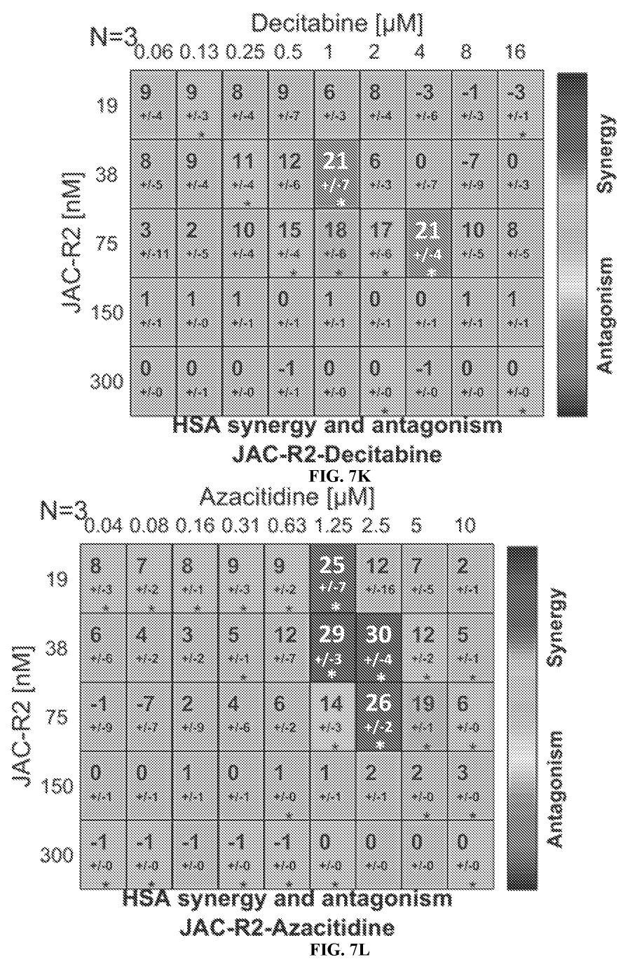

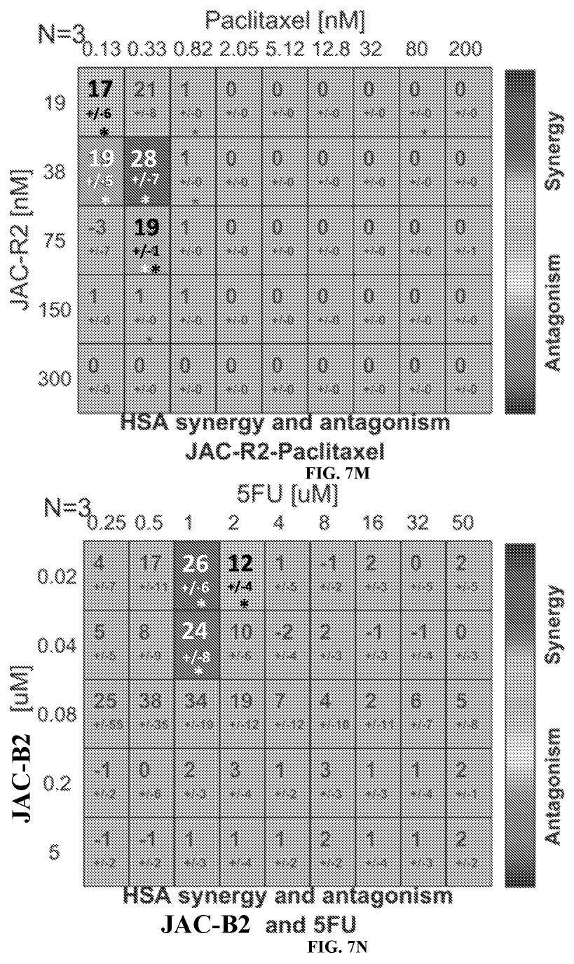

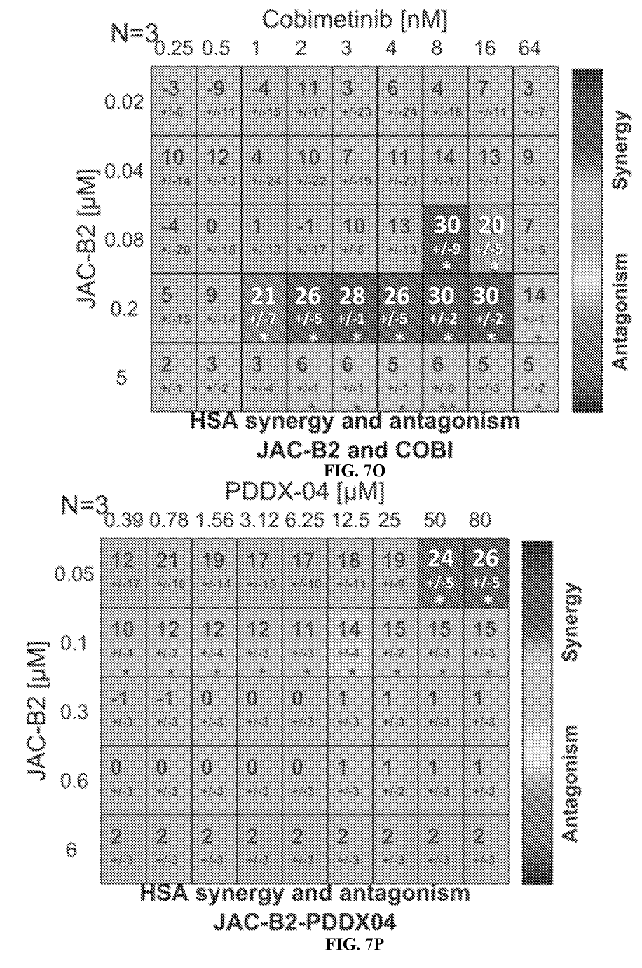

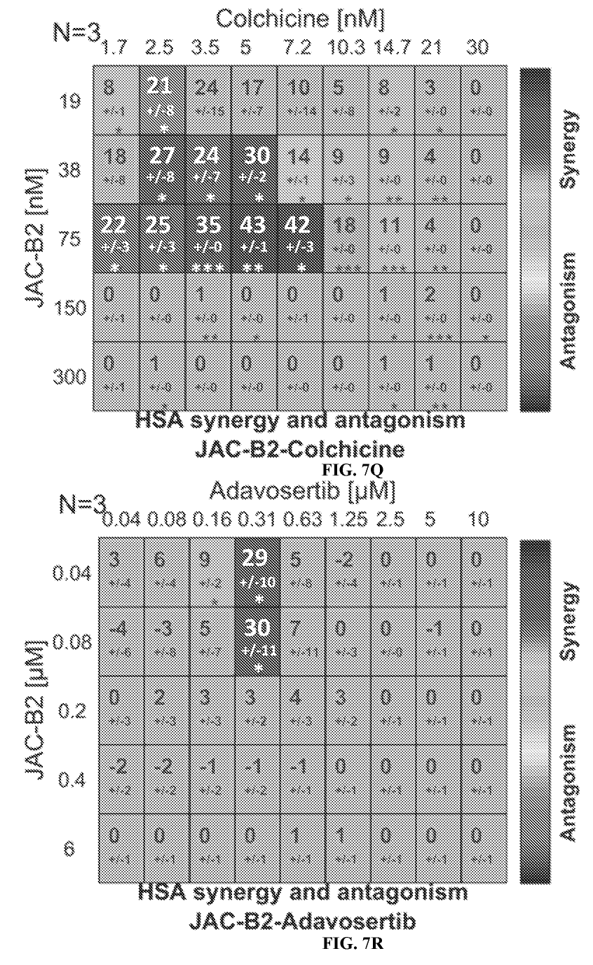

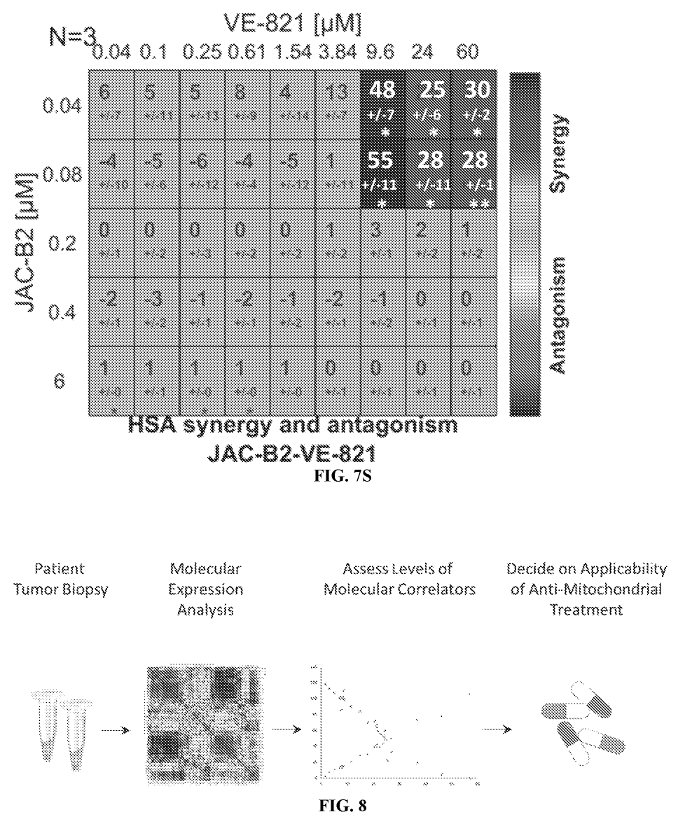

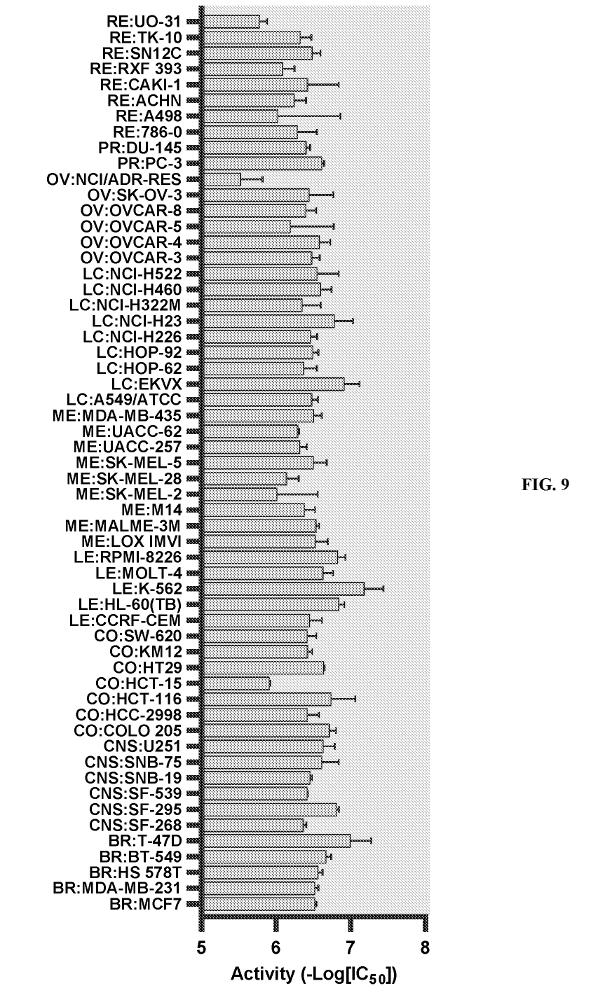

Efficacy of combinations of Compounds of Formula I (Compound 1) and an addition active agent (Compound 2)

| | | | |
|---|---|---|---|
| Compound 1 | Compound 2 | Effect | Supporting Data |
| JAC-B2 | Gemcitabine | Antagonistic/Additive | FIG. 7A |
| JAC-B2 | Methotrexate | Additive | FIG. 7B |
| JAC-B2 | Irinotecan | Additive | FIG. 7C |
| JAC-B2 | Olaparib | Antagonistic/Additive | FIG. 7D |
| JAC-B2 | Everolimus | Additive | FIG. 7E |
| JAC-B2 | Paclitaxel | Synergistic | FIG. 7F |
| JAC-B2 | Oxaliplatin | Synergistic | FIG. 7G |
| JAC-B2 | Abemaciclib | Synergistic | FIG. 7H |
| JAC-B2 | Decitabine | Synergistic | FIG. 7I |
| JAC-B2 | Azacitidine | Synergistic | FIG. 7J |
| JAC-R2 | Decitabine | Synergistic | FIG. 7K |
| JAC-R2 | Azacitidine | Synergistic | FIG. 7L |
| JAC-R2 | Paclitaxel | Synergistic | FIG. 7M |
| JAC-B2 | 5-Fluorouracil | Synergistic | FIG. 7N |
| JAC-B2 | Cobimetinib | Synergistic | FIG. 7O |
| JAC-B2 | PDDX04 | Synergistic | FIG. 7P |
| JAC-B2 | Hydralazine | Antagonistic/Additive | |
| JAC-B2 | Colchicine | Synergistic | FIG. 7Q |
| JAC-B2 | Metformin | Additive | |
| JAC-B2 | Adavosertib | Synergistic | FIG. 7R |
| JAC-B2 | VE-821 | Synergistic | FIG. 7S |

### Example 3

In various embodiments, the addition of a pharmaceutically acceptable excipient such as glycerol, bovine serum albumin, or sodium deoxycholate can improve the aqueous solubility of compounds of Formula I such that they are fully dissolved. Compounds of Formula I are generally sparingly soluble (1 g per 30-100 mL), slightly soluble (1 g in 100 to 1000 mL), very slightly soluble (1 g in 1 L to 10 L), or insoluble (1 g in more than 10 L) in water. The solubility definitions mean that at least the stated amount of water or other solvent is necessary to dissolve 1 g of a compound of Formula I. The following abbreviations are used in the tables below:

| | |
|---|---|
| Abbreviation | Meaning |
| LP | Large Particles |
| SP | Small Particle |
| MFG | Minimal Fine Grains |
| FD | Fully Dissolved |
| LS | Large Sediment |
| SS | Small Sediment |
| MS | Minimal Sediment |
| FD | Fully Dissolved |

Tables 3A, 3B, and 3C describe solubility experiments conducted with the indicated compounds under the listed conditions. Vortexing was conducted using a Denville Vortexer 59 (Denville Scientific Inc.), at maximum speed for 30 seconds. As used herein, the term overnight refers to a period lasting at least about 12-24 hours.

TABLE 3A

Solubility of glycerol-containing formulations post vortexing

| | | Post Vortexing | | | | | |
|---|---|---|---|---|---|---|---|
| Compound | Concentration | 0% | 10% | 20% | 30% | 40% | 50% |
| JAC-B1 | 2 mg/mL | LP | SP | SP | SP | SP | FD |
| JAC-B2 | 2 mg/mL | LP | LP | LP | LP | SP | FD |
| JAC-B2.5 | 2 mg/mL | LP | LP | LP | LP | L | MFG |
| JAC-R1 | 2 mg/mL | LP | LP | LP | LP | LP | SP |

TABLE 3B

Solubility of glycerol-containing formulations after 30 min incubation at 18° C.
After 30 minutes incubation at 18° C.

| Compound | Concentration | 0% | 10% | 20% | 30% | 40% | 50% |
|---|---|---|---|---|---|---|---|
| JAC-B1 | 2 mg/mL | SS | SS | SS | SS | SS | FD |
| JAC-B2 | 2 mg/mL | LS | LS | LS | LS | SS | SS |
| JAC-B2.5 | 2 mg/mL | LS | LS | LS | LS | LS | LS |
| JAC-R1 | 2 mg/mL | LS | LS | LS | LS | LS | SS |

TABLE 3C

Solubility of glycerol-containing formulations after overnight incubation at 18° C.

| | | Overnight incubation at 18° C. | | | | | |
|---|---|---|---|---|---|---|---|
| Compound | Concentration | 0% | 10% | 20% | 30% | 40% | 50% |
| JAC-B1 | 2 mg/mL | SS | SS | SS | SS | SS | MFG |
| JAC-B2 | 2 mg/mL | LS | LS | LS | LS | SS | SS |
| JAC-B2.5 | 2 mg/mL | LS | LS | LS | LS | LS | LS |
| JAC-R1 | 2 mg/mL | LS | LS | LS | LS | LS | SS |

Tables 4A, 4B, and 4C describe solubility experiments conducted with the indicated compounds under the listed conditions. Vortexing was conducted using a Denville Vortexer 59 (Denville Scientific Inc.), at maximum speed for 30 seconds. As used herein, the term overnight refers to a period lasting at least about 12-24 hours.

TABLE 4A

Solubility of BSA-containing formulations post vortexing

| | | Post Vortexing | | | | | | | |
|---|---|---|---|---|---|---|---|---|---|
| Cmpd. | Conc. | 5 mg/ mL | 10 mg/ mL | 15 mg/ mL | 25 mg/ mL | 40 mg/ mL | 60 mg/ mL | 80 mg/ mL | 100 mg/ mL |
| JAC-B 1 | 2 mg/mL | FD | FD | FD | FD | FD | FD | FD | FD |
| JAC-B2 | 2 mg/mL | | | | | LP | MFG | FD | FD |
| JAC-B2.5 | 2 mg/mL | | | | | LP | LP | LP | LP |
| JAC-R1 | 2 mg/mL | | | | | LP | LP | LP | MFG |

TABLE 4B

Solubility of BSA-containing formulations after 30 min incubation at 18° C.

| | | After 30 minutes incubation at 18° C. | | | | | | | |
|---|---|---|---|---|---|---|---|---|---|
| Cmpd. | Conc. | 5 mg/mL | 10 mg/mL | 15 mg/ mL | 25 mg/ mL | 40 mg/ mL | 60 mg/ mL | 80 mg/ mL | 100 mg/ mL |
| JAC-B1 | 2 mg/mL | SS | SS | SS | FD | | | FD | FD |
| JAC-B2 | 2 mg/mL | | | | | SS | SS | SS | SS |
| JAC-B2.5 | 2 mg/mL | | | | | LS | LS | LS | LS |
| JAC-R1 | 2 mg/mL | | | | | LS | LS | LS | LS |

Table 4C

Solubility of BSA-containing formulations after overnight incubation at 18° C.
Overnight incubation at 18° C.

| | | | | | | | | | |
|---|---|---|---|---|---|---|---|---|---|
| Cmpd. | Conc. | 5 mg/ mL | 10 mg/ mL | 15 mg/ mL | 25 mg/ mL | 40 mg/ mL | 60 mg/ mL | 80 mg/ mL | 100 mg/mL |
| JAC-B1 | 2 mg/mL | MFG | MFG | MFG | MFG | | | FD | FD |
| JAC-B2 | 2 mg/mL | | | | | LS | LS | MFG | MFG |
| JAC-B2.5 | 2 mg/mL | | | | | LS | LS | LS | LS |
| JAC-R1 | 2 mg/mL | | | | | SS | SS | SS | SS |

Tables 5A, 5B, and 5C describe solubility experiments conducted with the indicated compounds under the listed conditions. Vortexing was conducted using a Denville Vortexer 59 (Denville Scientific Inc.), at maximum speed for 30 seconds. As used herein, the term overnight refers to a period lasting at least about 12-24 hours.

TABLE 5A

Solubility of sodium deoxycholate-containing formulations post vortexing

| | | Post Vortexing | | | |
|---|---|---|---|---|---|
| Comp. | Conc. | 12.5 mg/mL | 25 mg/ mL | 50 mg/ mL | 100 mg/ mL |
| JAC-B1 | 2 mg/mL | SS | SP | MFG | MFG |
| JAC-B2 | 2 mg/mL | FD | FD | FD | FD |
| JAC-B1.5 | 2 mg/mL | SP | SP | SP | MFG |
| JAC-R1 | 2 mg/mL | SS | MFG | MFG | MFG |

TABLE 5B

Solubility of sodium deoxycholate-containing formulations after 30 min incubation at 18° C.

| | | After 30 minutes incubation at 18° C. | | | |
|---|---|---|---|---|---|
| Compound | Concentration | 12.5 mg/mL | 25 mg/ mL | 50 mg/ mL | 100 mg/ mL |
| JAC-B1 | 2 mg/mL | LS | SS | MFG | MFG |
| JAC-B2 | 2 mg/mL | FD | FD | FD | FD |
| JAC-B1.5 | 2 mg/mL | LS | SS | SS | SS |
| JAC-R1 | 2 mg/mL | LS | LS | MFG | MFG |

TABLE 5C

Solubility of sodium deoxycholate-containing formulations after overnight incubation at 18° C.

| | | Overnight incubation at 18° C. | | | |
|---|---|---|---|---|---|
| Compound | Concentration | 12.5 mg/mL | 25 mg/ mL | 50 mg/ mL | 100 mg/ mL |
| JAC-B1 | 2 mg/mL | SS | FD | FD | FD |
| JAC-B2 | 2 mg/mL | FD | FD | FD | FD |
| JAC-B1.5 | 2 mg/mL | LS | SS | SS | MFG |
| JAC-R1 | 2 mg/mL | FD | FD | FD | FD |

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following enumerated embodiments are provided, the numbering of which is not to be construed as designating levels of importance:
Embodiment 1 provides a method of treating, ameliorating, and/or preventing a cancer in a subject in need thereof, the method comprising:
   administering to the subject a composition comprising a therapeutically effective amount of a compound of Formula I, a pharmaceutically acceptable salt thereof, or a geometric isomer thereof: 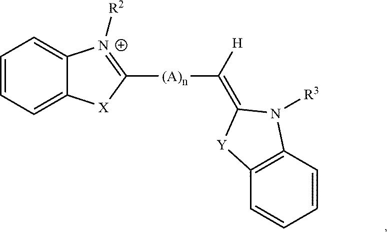
   wherein:
      X and Y are each independently selected from the group consisting of O, S, CH2, and C(CH₃)₂;
      A is 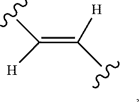
      , wherein one hydrogen atom in (A)ₙ is optionally substituted by R¹;
      R¹ is selected from the group consisting of -C₁₋₅ hydrocarbyl, -C₁₋₅-C(=O)OR, and -C₁₋₅ alkoxy;
      R₂ and R₃ are each independently selected from the group consisting of -C₁₋₅ hydrocarbyl, -C₁₋₅-C(=O)OR, and -C₁₋₅ alkoxy;
      each occurrence of R is independently H, C₁₋₄ alkyl, or C₆₋₁₀ aryl; and
      n is 0, 1, 2, or 3.
Embodiment 2 provides the method of embodiment 1, wherein the pharmaceutically acceptable salt is a halide salt.
Embodiment 3 provides the method of any one of embodiments 1-2, wherein the pharmaceutically acceptable salt is iodide.
Embodiment 4 provides the method of any one of embodiments 1-3, wherein the composition comprises an encapsulating agent.
Embodiment 5 provides the method of any one of embodiments 1-4, wherein the encapsulating agent comprises a nanoparticle or a liposome.
Embodiment 6 provides the method of any one of embodiments 1-5, wherein the encapsulating agent comprises a micelle, a nucleic-acid based dendrimer nanocarrier, a protein-based nanocarrier, a polymer-based nanoparticle, a lipid-polymer based nanoparticle, a metal-based nanoparticle, a nanocrystal, a liposomal carrier, a carbon nanotube based nanoparticle, or a polymeric micelle.
Embodiment 7 provides the method of any one of embodiments 1-6, wherein the encapsulating agent comprises mammalian serum albumin.
Embodiment 8 provides the method of any one of embodiments 1-7, wherein the mammalian serum albumin is of human origin.
Embodiment 9 provides the method of any one of embodiments 1-8, wherein the encapsulating agent further comprises a targeting agent.
Embodiment 10 provides the method of any one of embodiments 1-9, wherein the targeting agent comprises a peptide ligand, a nucleotide ligand, a polysaccharide ligand, a fatty acid ligand, a lipid ligand, a small molecule ligand, an antibody, an antibody fragment, an antibody mimetic, or an antibody mimetic fragment.
Embodiment 11 provides the method of any one of embodiments 1-10, wherein the targeting agent comprises folic acid.
Embodiment 12 provides the method of any one of embodiments 1-11, wherein the targeting agent comprises transferrin.
Embodiment 13 provides the method of any one of embodiments 1-12, wherein the composition is administered orally or parenterally.
Embodiment 14 provides the method of any one of embodiments 1-13, wherein the compound is not

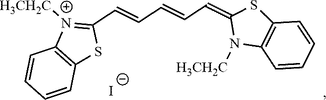

and
   the cancer is selected from group consisting of liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer.
Embodiment 15 provides the method of any one of embodiments 1-14, wherein the compound is

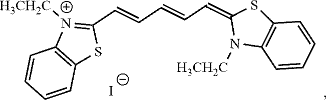

and
   the cancer is selected from the group consisting of liver cancer, rectal cancer, gastric cancer, esophageal cancer, anal cancer, non-melanoma skin cancer, small intestine cancer, bile duct cancer, periampullary cancer, endometrial cancer, cervical cancer, small cell lung cancer, sarcoma, neuroendocrine cancer, lymphoma, hepatoblastoma, Wilm’s tumor and primary mesothelial cancer.
Embodiment 16 provides the method of any one of embodiments 1-15, wherein the composition is administered at an advanced stage of peritoneal involvement by a cancer selected from the group consisting of liver cancer, rectal cancer, gastric cancer, esophageal cancer, head& neck cancer, renal cancer, breast cancer, ovarian cancer, endometrial cancer, cervical cancer, anal cancer, skin cancer, prostate cancer, small intestine cancer, bile duct cancer, pancreatic cancer, lung cancer, sarcoma, neuroendocrine cancer, and primary mesothelial cancer.
Embodiment 17 provides the method of any one of embodiments 1-16, wherein the neuroendocrine cancer is selected from the group consisting of adrenocortical carcinoma, a carcinoid tumor, a pancreatic neuroendocrine tumor, and thymic cancer.
Embodiment 18 provides the method of any one of embodiments 1-17, further comprising administering at least one other chemotherapeutic agent.
Embodiment 19 provides the method of embodiment 18, wherein the at least one other chemotherapeutic agent comprises a DNA damaging agent, a microtubule inhibitor, a Topoisomerase inhibitor, an anti-metabolite , a cell cycle checkpoint inhibitor, a mechanistic target of rapamycin kinase (mTOR) inhibitor, a receptor tyrosine kinase (RTK) inhibitor, a DNA damage repair inhibitor, a ATM or ATR inhibitor, a Non-Homologous End Joining (NHEJ) inhibitor, a Poly(ADP) ribose glycohydrolase (PARG) inhibitor, DNA Methyl Transfrase inhibitors (DNMT), an anti-cell surface protein, an immune checkpoint inhibitor, an anti-diabetic drug, an immune modulator, or a combination chemotherapy.
Embodiment 20 provides the method of any one of embodiments 18-19, wherein the DNA damaging agent comprises 5-Fluorouracil, Gemcitabine, Cytarabine, Capecitabine, Dacarbazine, Temozolomide, Mitomycin C, Paclitaxel, Docetaxel, nab-Paclitaxel, Ifosfamide, Cyclophosphamide, Adriamycin, Doxorubicin, Cisplatin, Carboplatin, or Oxaliplatin.
Embodiment 21 provides the method of any one of embodiments 18-20, wherein the microtubule inhibitor comprises Vincristine, Vinorelbine, or Vinblastine.
Embodiment 22 provides the method of any one of embodiments 18-21, wherein the topoisomerase inhibitor comprises Topotecan, Irinotecan, or Etoposide.
Embodiment 23 provides the method of any one of embodiments 18-22, wherein the CDK inhibitor comprises an inhibitor of CDK4, an inhibitor of CDK6 or an inhibitor of CDK4 and CDK6.
Embodiment 24 provides the method of any one of embodiments 18-23, wherein the DNA damage repair inhibitor is Olaparib, Veliparib, or Talazoparib, Rucaparib, Niraparib.
Embodiment 25 provides the method of any one of embodiments 18-24, wherein the Cell cycle checkpoint inhibitor is a WEE1 inhibitor.
Embodiment 26 provides the method of any one of embodiments 18-25, wherein the DNMT inhibitor is a Azacytadine, Decitabine, Glyburide, Panobinostat.
Embodiment 27 provides the method of any one of embodiments 18-26, wherein the anti-diabetic drug is Metformin, Acarbose, Miglitol, a Sodium-glucose transporter (SGLT) inhibitor, or a Thiazolidinedione.
Embodiment 28 provides the method of any one of embodiments 18-27, wherein the ATR and/or ATM inhibitor is VE-821, M6620, AZD1390 or BAY-1895344.
Embodiment 29 provides the method of any one of embodiments 1-28, wherein the method further comprises administering at least one hormone antagonist, selective hormone receptor modulating agent, or hormone enzyme inhibitor.
Embodiment 30 provides the method of any one of embodiments 1-30, wherein the method further comprises radiation therapy.
Embodiment 31 provides the method of any one of embodiments 1-31, wherein the method of treatment alleviates a symptom of the cancer.
Embodiment 32 provides the method of any one of embodiments 1-31, wherein the subject is human.
Embodiment 33 provides the method of any one of embodiments 1-32, wherein the subject has a plasma compound concentration about 0.2 ng/mL to about 20 ng/mL at about 1 hour after administration of the composition.
Embodiment 34 provides the method of any one of embodiments 1-33, wherein the subject has a plasma compound concentration of about 0.5 ng/mL to about 50 ng/mL at about 4 hours after administration of the composition.
Embodiment 35 provides the method of any one of embodiments 1-34, wherein the subject has a plasma compound concentration of about 0.5 ng/mL to about 50 ng/mL at up to about 5 hours after administration of the composition.
Embodiment 36 provides the method of any one of embodiments 1-35, wherein the subject has a concentration of the compound in the pancreas of about 5 ng/mL to about 100 ng/mL at about 4 hours after administration of the composition.
Embodiment 37 provides the method of any one of embodiments 1-36, wherein the subject has a concentration of the compound concentration in the pancreas of about 1 ng/mL to about 100 ng/mL at about 8 hours after administration of the composition.
Embodiment 38 provides the method of any one of embodiments 1-37, wherein the subject has a concentration of the compound concentration in the pancreas of about 100 ng/mL to about 400 ng/mL at up to about 8 hours after administration of the composition.
Embodiment 39 provides a compound of Formula I, a pharmaceutically acceptable salt thereof, or a geometric isomer thereof:

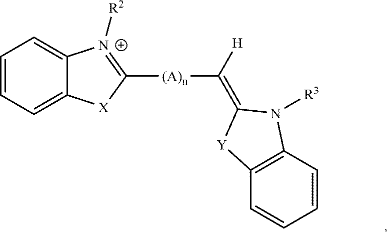

wherein:
   X and Y are each independently selected from the group consisting of O, S, CH₂, and C(CH₃)₂;
   A is 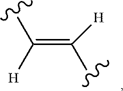
   wherein one hydrogen atom in (A)ₙ is optionally substituted by R¹;
   R¹ is selected from the group consisting of H, -C₁₋₅ hydrocarbyl, -C₁₋₅-C(=O)OR, and -C₁₋₅ alkoxy;
   R₂ and R₃ are each independently selected from the group consisting of -C₁₋₅ hydrocarbyl, -C₁₋₅-C(=O)OR, and -C₁₋₅ alkoxy;
   each occurrence of R is independently H, C₁₋₄ alkyl, or C₆₋₁₀ aryl; and
   n is 0, 1, 2, or 3,
   with the proviso that the compound is not: 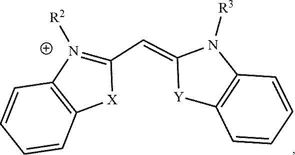
   X and Y are S and R² and R³ are methyl or ethyl; 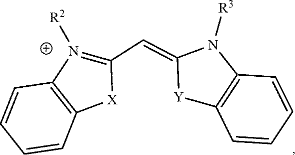
   X and Y are O and R² and R³ are ethyl; 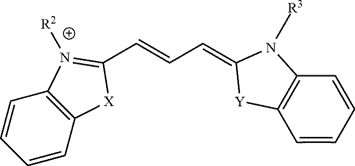
   X and Y are S, and R² and R³ are identical and are C₁₋₆ hydrocarbons; 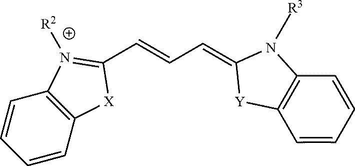
   X and Y are O, and R² and R³ are identical and are C₁₋₆ hydrocarbons; 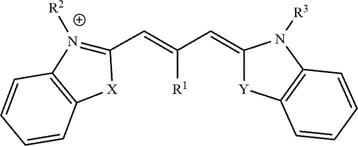
   X and Y are S, R¹ is methyl, and R² and R³ are methyl, ethyl or butyl; 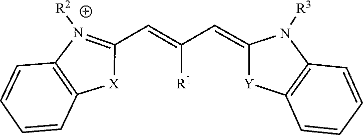
   X and Y are S, R¹ is ethyl, and R² and R³ are identical and are C₁₋₄ hydrocarbons; 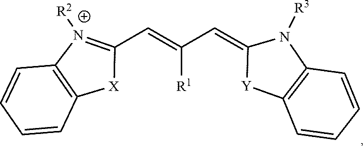
   X and Y are O, R¹ is H, and R² and R³ are identical and are C₁₋₆ hydrocarbons; 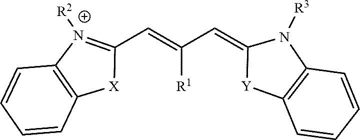
   X and Y are O, R¹ is methyl, and R² and R³ are ethyl; 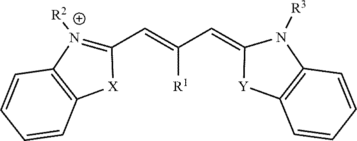
   X and Y are O, R¹ is ethyl, and R² and R³ are identical and are methyl or ethyl; 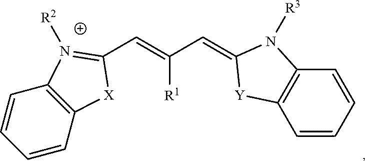
   X and Y are C(CH₃)₂, R¹ is H, and R² and R³ are identical and are C₁₋₆ hydrocarbons; 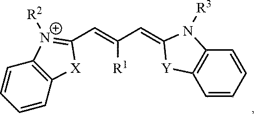
   X and Y are C(CH₃)₂, R¹ is methyl, and R² and R³ are ethyl; 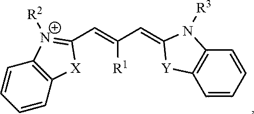
   X and Y are C(CH₃)₂, R¹ is ethyl, and R² and R³ are identical and are methyl or ethyl; 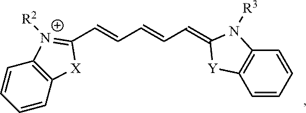
   X and Y are S, and R² and R³ are identical and are C₁-₅ hydrocarbons; 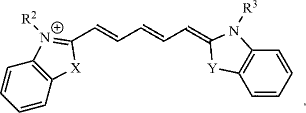
   X and Y are O, and R² and R³ are identical and are ethyl, propyl, or pentyl; 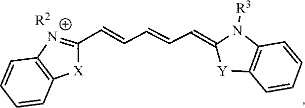
   X and Y are C(CH₃)₂, and R² and R³ are identical and are methyl, ethyl, or propyl; 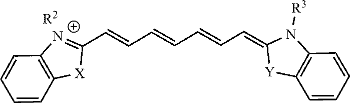
   X and Y are O, and R² and R³ are identical and are methyl or ethyl; 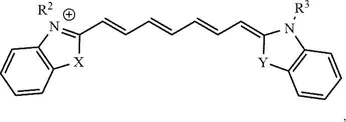
   X and Y are S, and R² and R³ are identical and are C₁-₅ hydrocarbons; or 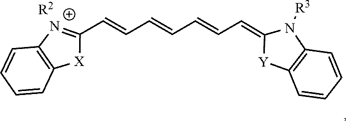
   X and Y are C(CH₃)₂, and R² and R³ are identical and are methyl or ethyl.
Embodiment 40 provides the compound of embodiment 39, wherein the pharmaceutically acceptable salt is a halide salt.
Embodiment 41 provides the compound of any one of embodiments 39-40, wherein the pharmaceutically acceptable salt is iodide.
Embodiment 42 provides the compound of any one of embodiments 39-41, wherein R¹ is H.
Embodiment 43 provides the compound of any one of embodiments 39-42, wherein R² and R³ are -C₁₋₅-C(=O)OR.
Embodiment 44 provides the compound of any one of embodiments 39-43, wherein R² and R³ are -(CH₂)₄-C(=O)OR.
Embodiment 45 provides the compound of any one of embodiments 39-44, wherein n is 0.
Embodiment 46 provides a compound of Formula Ia, a pharmaceutically acceptable salt thereof, or a geometric isomer thereof:

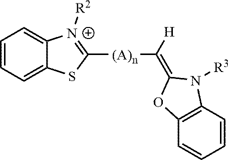

wherein:
   A is 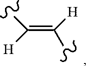
   wherein one hydrogen atom in (A)ₙ is optionally substituted by R¹;
   R¹ is selected from the group consisting of H, -C₁₋₅ hydrocarbyl, -C₁₋₅-C(=O)OR, and -C₁₋₅ alkoxy;
   R² and R₃ are each independently selected from the group consisting of -C₁₋₅ hydrocarbyl, -C₁₋₅-C(=O)OR, and -C₁₋₅ alkoxy;
   each occurrence of R is independently H, C₁₋₄ alkyl, or C₆₋₁₀ aryl; and
   n is 0, 1, 2, or 3.
Embodiment 47 provides the compound of any one of embodiments 39-46, wherein R² and R³ are ethyl.
Embodiment 48 provides a method for predicting cancer drug response in a subject, the method comprising:
   obtaining the results of molecular data from a subject’s tissue sample, using a response prediction model on the data and applying results of the model to select a particular treatment for cancer, wherein the particular treatment comprises a treatment with a compound of Formula I or a pharmaceutically acceptable salt or geometric isomer thereof, wherein the correlation is obtained by:
      i. identifying, based on gene, protein or metabolic expression levels, at least one gene product, protein, metabolite, cellular hallmark pathway, or one cellular genetic pathway involved in mitochondrial activity; and
      ii. creating a regression or correlation model derived from experimental activity data of the compound of Formula I on the at least one cellular pathway or gene product in the subject’s tissue sample,
      wherein the compound of Formula I, has the structure: 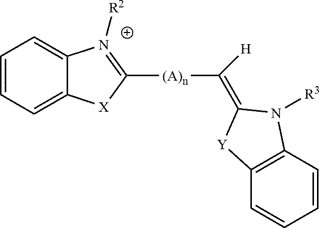
   wherein:
      X and Y are each independently selected from the group consisting of O, S, CH2, and C(CH₃)₂;
      A is 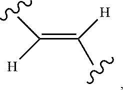
      wherein one hydrogen atom in (A)ₙ is optionally substituted by R¹;
      R¹ is selected from the group consisting of -C₁₋₅ hydrocarbyl, -C₁₋₅-C(=O)OR, and -C₁₋₅ alkoxy;
      R² and R₃ are each independently selected from the group consisting of -C₁₋₅ hydrocarbyl, -C₁₋₅-C(=O)OR, and -C₁₋₅ alkoxy;
      each occurrence of R is independently H, C₁₋₄ alkyl, or C₆₋₁₀ aryl; and
      n is 0, 1, 2, or 3.
Embodiment 49 provides the method of embodiment 48, wherein the treatment response prediction is used to treat cancer in the subject by obtaining molecular data from the subject’s tissue sample; and
   treating the subject with the compound of Formula I if the response model predicts a potency of the compound of Formula I on the tissue sample above a predetermined threshold level.
Embodiment 50 provides the method of any one of embodiments 48-49, wherein the predetermined threshold level is a predicted -log(IC₅₀) of at least 5.0.
Embodiment 51 provides the method of any one of embodiments 48-50, wherein the model is based on experimental activity of at least one compound of Formula I on the a gene set containing at least, one gene target or signature from Tables 1A-1C.
Embodiment 52 provides the method of any one of embodiments 48-51, wherein the model is based on experimental activity of at least one compound of Formula I on a molecular expression model containing at least the MYO1B and CPT1A genes.
Embodiment 53 provides the method of any one of embodiments 48-52, wherein the compound of Formula I is a halide salt of

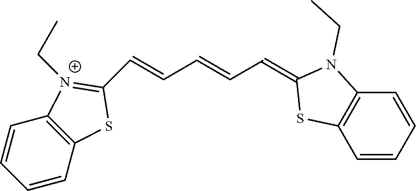 [SMILES: CCN1/C(=C/C=C/C=C/c2sc3ccccc3[n+]2CC)Sc2ccccc21]

Embodiment 54 provides the method of any one of embodiments 48-53, wherein the halide salt is iodide.
Embodiment 55 provides the method of any one of embodiments 48-54, wherein the compound is not

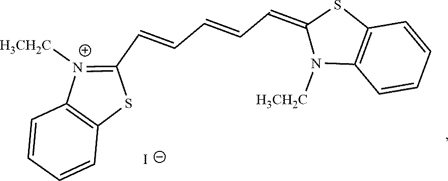

and
   the cancer is selected from group consisting of liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer.
Embodiment 56 provides the method of any one of embodiments 48-55, wherein the compound is

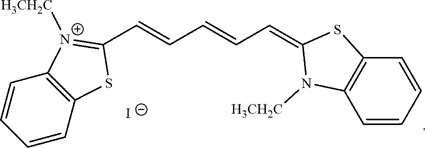

and
   the cancer is selected from the group consisting of liver cancer, rectal cancer, gastric cancer, esophageal cancer, anal cancer, non-melanoma skin cancer, small intestine cancer, bile duct cancer, periampullary cancer, endometrial cancer, cervical cancer, small cell lung cancer, sarcoma, neuroendocrine cancer, lymphoma, hepatoblastoma, Wilm’s tumor and primary mesothelial cancer.
Embodiment 57 provides a method of treating, ameliorating, and/or preventing a cancer in a subject in need thereof, the method comprising:
   administering to the subject a composition comprising a therapeutically effective amount of a compound of Formula II, a pharmaceutically acceptable salt thereof, or a geometric isomer thereof: 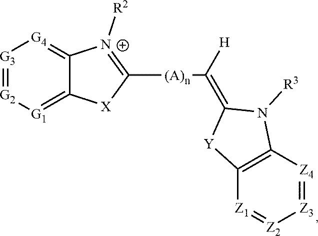
   wherein:
      each of G₁, G₂, G₃, and G₄ is independently C(=O), CR′, CNR″₂, N, or N-R″;
      each of Z₁, Z₂, Z₃, and Z₄ is independently C(=O), CR′, CNR″₂, N, or N-R″;
      X and Y are each independently selected from the group consisting of O, N, S, CH2, and C(CH₃)₂;
      A is 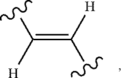
      wherein one hydrogen atom in (A)ₙ is optionally substituted by R¹; R¹ is selected from the group consisting of -C₁₋₅ hydrocarbyl, -C₁₋₅-C(=O)OR, and -C₁₋₅ alkoxy;
      R₂ and R₃ are each independently selected from the group consisting of-C₁₋₅ hydrocarbyl, -C₁₋₅-C(=O)OR, and -C₁₋₅ alkoxy;
      each occurrence of R′ is independently H or C₁₋₄ alkyl,
      each occurrence of R″ is independently H or C₁₋₄ alkyl;
      each occurrence of R is independently H, C₁₋₄ alkyl, or C₆₋₁₀ aryl; and
      n is 0, 1, 2, or 3.
Embodiment 58 provides the method of embodiment 57, wherein the pharmaceutically acceptable salt is a halide salt.
Embodiment 59 provides the method of any one of embodiments 57-58, wherein the pharmaceutically acceptable salt is iodide.
Embodiment 60 provides the method of any one of embodiments 57-59, wherein the composition comprises an encapsulating agent.
Embodiment 61 provides the method of any one of embodiments 57-60, wherein the encapsulating agent comprises a nanoparticle or a liposome.
Embodiment 62 provides the method of any one of embodiments 57-61, wherein the encapsulating agent comprises a micelle, a nucleic-acid based dendrimer nanocarrier, a protein-based nanocarrier, a polymer-based nanoparticle, a lipid-polymer based nanoparticle, a metal-based nanoparticle, a nanocrystal, a liposomal carrier, a carbon nanotube based nanoparticle, or a polymeric micelle.
Embodiment 63 provides the method of any one of embodiments 57-62, wherein the encapsulating agent comprises mammalian serum albumin.
Embodiment 64 provides the method of any one of embodiments 57-63, wherein the mammalian serum albumin is of human origin.
Embodiment 65 provides the method of any one of embodiments 57-63, wherein the encapsulating agent further comprises a targeting agent.
Embodiment 66 provides the method of any one of embodiments 57-64, wherein the targeting agent comprises a peptide ligand, a nucleotide ligand, a polysaccharide ligand, a fatty acid ligand, a lipid ligand, a small molecule ligand, an antibody, an antibody fragment, an antibody mimetic, or an antibody mimetic fragment.
Embodiment 67 provides the method of any one of embodiments 57-65, wherein the targeting agent comprises folic acid.
Embodiment 68 provides the method of any one of embodiments 57-66, wherein the targeting agent comprises transferrin.
Embodiment 69 provides the method of any one of embodiments 57-68, wherein the composition is administered orally or parenterally.
Embodiment 70 provides the method of any one of embodiments 57-69, wherein the cancer is selected from group consisting of liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer.
Embodiment 71 provides the method of any one of embodiments 57-70, wherein the composition is administered at an advanced stage of peritoneal involvement by a cancer selected from the group consisting of liver cancer, rectal cancer, gastric cancer, esophageal cancer, head& neck cancer, renal cancer, breast cancer, ovarian cancer, endometrial cancer, cervical cancer, anal cancer, skin cancer, prostate cancer, small intestine cancer, bile duct cancer, pancreatic cancer, lung cancer, sarcoma, neuroendocrine cancer, and primary mesothelial cancer.
Embodiment 72 provides the method of any one of embodiments 57-71, wherein the neuroendocrine cancer is selected from the group consisting of adrenocortical carcinoma, a carcinoid tumor, a pancreatic neuroendocrine tumor, and thymic cancer.
Embodiment 73 provides the method of any one of embodiments 57-72, further comprising administering at least one other chemotherapeutic agent.
Embodiment 74 provides the method of embodiment 73, wherein the at least one other chemotherapeutic agent comprises a DNA damaging agent, a microtubule inhibitor, a Topoisomerase inhibitor, an anti-metabolite , a cell cycle checkpoint inhibitor, a mechanistic target of rapamycin kinase (mTOR) inhibitor, a receptor tyrosine kinase (RTK) inhibitor, a DNA damage repair inhibitor, a ATM or ATR inhibitor, a Non-Homologous End Joining (NHEJ) inhibitor, a Poly(ADP) ribose glycohydrolase (PARG) inhibitor, DNA Methyl Transfrase inhibitors (DNMT), an anti-cell surface protein, an immune checkpoint inhibitor, an anti-diabetic drug, an immune modulator, or a combination chemotherapy.
Embodiment 75 provides the method of any one of embodiments 73-74, wherein the DNA damaging agent comprises 5-Fluorouracil, Gemcitabine, Cytarabine, Capecitabine, Dacarbazine, Temozolomide, Mitomycin C, Paclitaxel, Docetaxel, nab-Paclitaxel, Ifosfamide, Cyclophosphamide, Adriamycin, Doxorubicin, Cisplatin, Carboplatin, or Oxaliplatin.
Embodiment 76 provides the method of any one of embodiments 73-74, wherein the microtubule inhibitor comprises Vincristine, Vinorelbine, or Vinblastine.
Embodiment 77 provides the method of any one of embodiments 73-76, wherein the topoisomerase inhibitor comprises Topotecan, Irinotecan, or Etoposide.
Embodiment 78 provides the method of any one of embodiments 73-77, wherein the CDK inhibitor comprises an inhibitor of CDK4, an inhibitor of CDK6 or an inhibitor of CDK4 and CDK6.
Embodiment 79 provides the method of any one of embodiments 73-78, wherein the DNA damage repair inhibitor is Olaparib, Veliparib, or Talazoparib, Rucaparib, Niraparib.
Embodiment 80 provides the method of any one of embodiments 73-79, wherein the Cell cycle checkpoint inhibitor is a WEE1 inhibitor.
Embodiment 81 provides the method of any one of embodiments 73-80, wherein the DNMT inhibitor is a Azacytadine, Decitabine, Glyburide, Panobinostat.
Embodiment 82 provides the method of any one of embodiments 73-81, wherein the anti-diabetic drug is Metformin, Acarbose, Miglitol, a Sodium-glucose transporter (SGLT) inhibitor, or a Thiazolidinedione.
Embodiment 83 provides the method of any one of embodiments 73-82, wherein the ATR and/or ATM inhibitor is VE-821, M6620, AZD1390 or BAY-1895344.
Embodiment 84 provides the method of any one of embodiments 57-83, wherein the method further comprises administering at least one hormone antagonist, selective hormone receptor modulating agent, or hormone enzyme inhibitor.
Embodiment 85 provides the method of any one of embodiments 57-84, wherein the method further comprises radiation therapy.
Embodiment 86 provides the method of any one of embodiments 57-85, wherein the method of treatment alleviates a symptom of the cancer.
Embodiment 87 provides a compound of Formula II, or a pharmaceutically acceptable salt thereof, or a geometric isomer thereof:

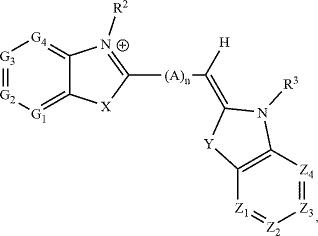

wherein:
   each of G₁, G₂, G₃, and G₄ is independently C(=O), CR′, CNR″₂, N, or N-R″;
   each of Z₁, Z₂, Z₃, and Z₄ is independently C(=O), CR′, CNR″₂, N, or N-R″;
   X and Y are each independently selected from the group consisting of O, N, S, CH₂, and C(CH₃)₂;
   A is 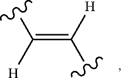
   wherein one hydrogen atom in (A)ₙ is optionally substituted by R¹;
   R¹ is selected from the group consisting of -C₁₋₅ hydrocarbyl, -C₁₋₅-C(=O)OR, and -C₁-₅ alkoxy;
   R₂ and R₃ are each independently selected from the group consisting of -C₁₋₅ hydrocarbyl, -C₁₋₅-C(=O)OR, and -C₁₋₅ alkoxy;
   each occurrence of R′ is independently H or C₁₋₄ alkyl;
   each occurrence of R″ is independently H or C₁₋₄ alkyl;
   each occurrence of R is independently H, C₁₋₄ alkyl, or C₆₋₁₀ aryl; and
   n is 0, 1, 2, or 3.
Embodiment 88 provides the compound of embodiment 87, wherein the compound is selected from the group consisting of

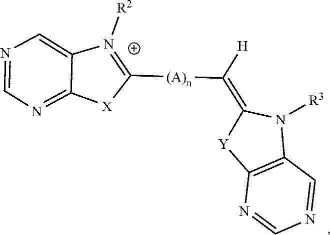

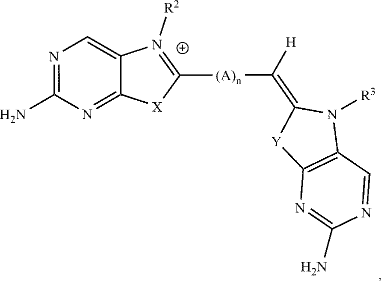

and

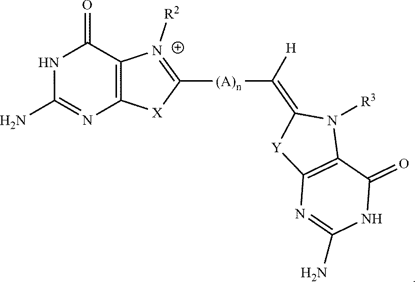

Embodiment 89 provides a method for predicting cancer drug response in a subject, the method comprising:
   obtaining the results of molecular data from a subject’s tissue sample, using a response prediction model on the data and applying results of the model to select a particular treatment for cancer, wherein the particular treatment comprises a treatment with a compound of Formula II or a pharmaceutically acceptable salt or geometric isomer thereof, wherein the correlation is obtained by:
      i. identifying, based on gene, protein or metabolic expression levels, at least one gene product, protein, metabolite, cellular hallmark pathway, or one cellular genetic pathway involved in mitochondrial activity; and
      ii. creating a regression or correlation model derived from experimental activity data of the compound of Formula II on the at least one cellular pathway or gene product in the subject’s tissue sample,
      wherein the compound of Formula II, has the structure: 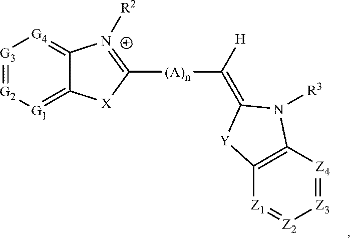
   wherein:
      each of G₁, G₂, G₃, and G₄ is independently C(=O), CR′, CNR″₂, N, or N-R″;
      each of Z₁, Z₂, Z₃, and Z₄ is independently C(=O), CR′, CNR″₂, N, or N-R″;
      X and Y are each independently selected from the group consisting of O, N, S, CH₂, and C(CH₃)₂;
      A is 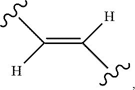
      wherein one hydrogen atom in (A)ₙ is optionally substituted by R¹;
      R¹ is selected from the group consisting of -C₁₋₅ hydrocarbyl, -C₁₋₅-C(=O)OR, and -C₁-₅ alkoxy;
      R₂ and R₃ are each independently selected from the group consisting of -C₁₋₅ hydrocarbyl, -C₁₋₅-C(=O)OR, and -C₁₋₅ alkoxy;
      each occurrence of R′ is independently H or C₁₋₄ alkyl;
      each occurrence of R″ is independently H or C₁₋₄ alkyl;
      each occurrence of R is independently H, C₁₋₄ alkyl, or C₆₋₁₀ aryl; and
      n is 0, 1, 2, or 3.
Embodiment 90 provides the method of embodiment 89, wherein the treatment response prediction is used to treat cancer in the subject by obtaining molecular data from the subject’s tissue sample; and
   treating the subject with the compound of Formula II if the response model predicts a potency of the compound of Formula II on the tissue sample above a predetermined threshold level.
Embodiment 91 provides the method of any one of embodiments 89-90, wherein the predetermined threshold level is a predicted -log(IC₅₀) of at least 5.0.
Embodiment 92 provides the method of any one of embodiments 89-91, wherein the model is based on experimental activity of at least one compound of Formula I on the a gene set containing at least, one gene target or signature from Tables 1A-1C.
Embodiment 93 provides the method of any one of embodiments 89-92, wherein the model is based on experimental activity of at least one compound of Formula I on a molecular expression model containing at least the MYO1B and CPT1A genes.

1. A method of treating, ameliorating, or preventing a cancer in a subject in need thereof, the method comprising:
administering to the subject a therapeutically effective amount of a compound of Formula I, a pharmaceutically acceptable salt thereof, or a geometric isomer thereof: 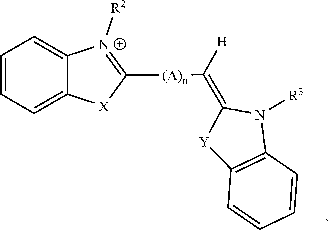
wherein:
X and Y are each independently selected from the group consisting of O, S, CH₂, and C(CH₃)₂;
A is 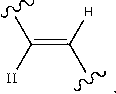
wherein one hydrogen atom in (A)ₙ is optionally substituted by R¹;
R¹ is selected from the group consisting of C₁₋₅ hydrocarbyl, C₁₋₅ hydrocarbyl-C(=O)OR, and C₁₋₅ alkoxy;
R² and R³ are each independently selected from the group consisting of C₁₋₅ hydrocarbyl, C₁₋₅ hydrocarbyl-C(=O)OR, and C₁₋₅ alkoxy;
each occurrence of R is independently H, C₁₋₄ alkyl, or C₆₋₁₀ aryl; and
n is 0, 1, 2, or 3;
optionally wherein the compound is administered to the subject as part of a composition, which is optionally formulated for oral or parenteral administration to the subject.

2. The method of claim 1, wherein the pharmaceutically acceptable salt is a halide salt, which is optionally an iodide salt.

3. (canceled)

4. The method of claim 1, wherein the composition comprises an encapsulating agent, optionally wherein at least one of the following applies:
(a) the encapsulating agent comprises a nanoparticle or a liposome;
(b) the encapsulating agent comprises a micelle, a nucleic-acid based dendrimer nanocarrier, a protein-based nanocarrier, a polymer-based nanoparticle, a lipid-polymer based nanoparticle, a metal-based nanoparticle, a nanocrystal, a liposomal carrier, a carbon nanotube based nanoparticle, or a polymeric micelle;
(c) the encapsulating agent comprises mammalian serum albumin, which is optionally of human origin;
(d) the encapsulating agent comprises a targeting agent, which optionally comprises at least one of the following:
(i) a peptide ligand, a nucleotide ligand, a polysaccharide ligand, a fatty acid ligand, a lipid ligand, a small molecule ligand, an antibody, an antibody fragment, an antibody mimetic, or an antibody mimetic fragment,
(ii) folic acid or transferrin.

5-13. (canceled)

14. The method of claim 1, wherein the compound is not 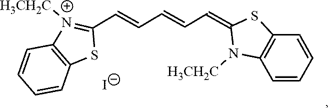
and
the cancer is selected from group consisting of liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer.

15. The method of claim 1, wherein the compound is 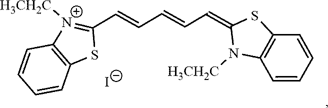
and
the cancer is selected from the group consisting of liver cancer, rectal cancer, gastric cancer, esophageal cancer, anal cancer, non-melanoma skin cancer, small intestine cancer, bile duct cancer, periampullary cancer, endometrial cancer, cervical cancer, small cell lung cancer, sarcoma, neuroendocrine cancer, lymphoma, hepatoblastoma, Wilm’s tumor and primary mesothelial cancer.

16. The method of claim 1, wherein the compound is administered at an advanced stage of peritoneal involvement by a cancer selected from the group consisting of liver cancer, rectal cancer, gastric cancer, esophageal cancer, head& neck cancer, renal cancer, breast cancer, ovarian cancer, endometrial cancer, cervical cancer, anal cancer, skin cancer, prostate cancer, small intestine cancer, bile duct cancer, pancreatic cancer, lung cancer, sarcoma, neuroendocrine cancer, and primary mesothelial cancer,
optionally wherein the neuroendocrine cancer is selected from the group consisting of adrenocortical carcinoma, a carcinoid tumor, a pancreatic neuroendocrine tumor, and thymic cancer.

17. (canceled)

18. The method of claim 1, further comprising administering to the subject at least one other chemotherapeutic agent,
optionally wherein the at least one other chemotherapeutic agent comprises a DNA damaging agent, a microtubule inhibitor, a Topoisomerase inhibitor, an anti-metabolite , a cell cycle checkpoint inhibitor, a mechanistic target of rapamycin kinase (mTOR) inhibitor, a receptor tyrosine kinase (RTK) inhibitor, a DNA damage repair inhibitor, a ATM or ATR inhibitor, a Non-Homologous End Joining (NHEJ) inhibitor, a Poly(ADP) ribose glycohydrolase (PARG) inhibitor, DNA Methyl Transfrase inhibitors (DNMT), an anti-cell surface protein, an immune checkpoint inhibitor, an anti-diabetic drug, an immune modulator, or a combination chemotherapy.

19. (canceled)

20. The method of claim 19, wherein at least of the following applies:
(a) the DNA damaging agent comprises 5-Fluorouracil, Gemcitabine, Cytarabine, Capecitabine, Dacarbazine, Temozolomide, Mitomycin C, Paclitaxel, Docetaxel, nab-Paclitaxel, Ifosfamide, Cyclophosphamide, Adriamycin, Doxorubicin, Cisplatin, Carboplatin, or Oxaliplatin,
(b) the microtubule inhibitor comprises Vincristine, Vinorelbine, or Vinblastine;
(c) the topoisomerase inhibitor comprises Topotecan, Irinotecan, or Etoposide,
(d) the CDK inhibitor comprises an inhibitor of CDK4, an inhibitor of CDK6, or an inhibitor of CDK4 and CDK6;
(e) the DNA damage repair inhibitor comprises Olaparib, Veliparib, Talazoparib, Rucaparib, or Niraparib;
(f) the cell cycle checkpoint inhibitor comprises a WEE1 inhibitor,
(g) the DNMT inhibitor comprises a Azacytadine, Decitabine, Glyburide, or Panobinostat;
(h) the anti-diabetic drug comprises Metformin, Acarbose, Miglitol, a Sodium-glucose transporter (SGLT) inhibitor, or a Thiazolidinedione;
(i) the ATR or ATM inhibitor comprises VE-821, M6620, AZD1390, or BAY-1895344.

21-28. (canceled)

29. The method of claim 1, wherein at least one of the following applies:
(a) the method further comprises administering to the subject at least one hormone antagonist, selective hormone receptor modulating agent, or hormone enzyme inhibitor,
(b) the method further comprises administering radiation therapy to the subject;
(c) the method alleviates a symptom of the subject’s cancer,
(d) the subject is human.

30-32. (canceled)

33. The method of claim 1, wherein at least one the following applies:
(a) the subject has a compound plasma concentration about 0.2 ng/mL to about 20 ng/mL at about 1 hour after administration of the compound
(b) the subject has a compound plasma concentration of about 0.5 ng/mL to about 50 ng/mL at about 4 hours after administration of the compound,
(c) the subject has a compound plasma concentration of about 0.5 ng/mL to about 50 ng/mL at up to about 5 hours after administration of the compound;
(d) the subject has a compound concentration of about 5 ng/mL to about 100 ng/mL in the pancreas at about 4 hours after administration of the compound;
(e) the subject has a compound concentration of about 1 ng/mL to about 100 ng/mL in the pancreas at about 8 hours after administration of the compound;
(f) the subject has a compound concentration of about 100 ng/mL to about 400 ng/mL in the pancreas at up to about 8 hours after administration of the compound.

34-38. (canceled)

39. A compound of Formula I, a pharmaceutically acceptable salt thereof, or a geometric isomer thereof: 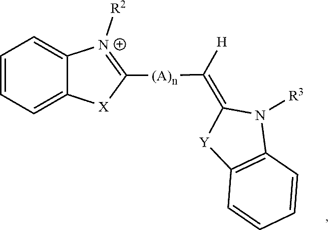
wherein:
X and Y are each independently selected from the group consisting of O, S, CH₂, and C(CH₃)₂;
A is 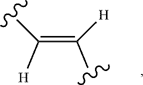
wherein one hydrogen atom in (A)ₙ is optionally substituted by R¹;
R¹ is selected from the group consisting of H, C₁-₅ hydrocarbyl, C₁₋₅ hydrocarbyl-C(=O)OR. and C₁₋₅ alkoxy;
R² and R³ are each independently selected from the group consisting of C₁₋₅ hydrocarbyl, C₁₋₅ hydrocarbyl-C(==O)OR, and C₁₋₅ alkoxy;
each occurrence of R is independently H, C₁₋₄ alkyl, or C₆₋₁₀ aryl; and
n is 0, 1, 2, or 3, and
optionally wherein the pharmaceutically acceptable salt is a halide salt;
with the proviso that the compound is not: 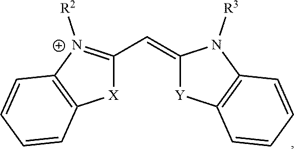
X and Y are S, and R² and R³ are methyl or ethyl; 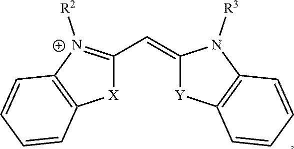
X and Y are O, and R² and R ³ are ethyl; 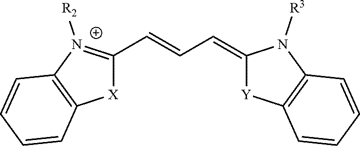
hydrocarbons; X and Y are S, and R² and R³ are identical and are C₁₋₆ 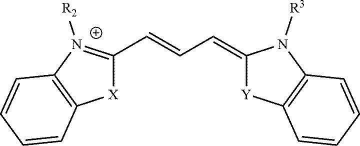
X and Y are O, and R² and R³ are identical and are C₁₋₆ hydrocarbons; 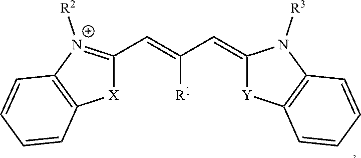
X and Y are S, R¹ is methyl, and R² and R ³ are methyl, ethyl or butyl; 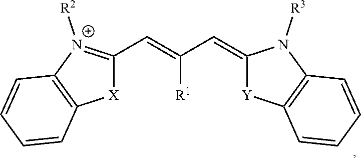
X and Y are S, R ¹ is ethyl, and R² and R³ are identical and are C₁₋₄ hydrocarbons; 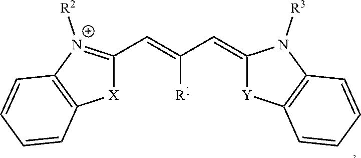
X and Y are O, R¹ is H, and R² and R³ are identical and are C₁₋₆ hydrocarbons; 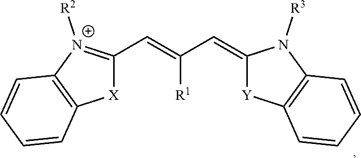
X and Y are O, R¹ is methyl, and R² and R³ are ethyl; 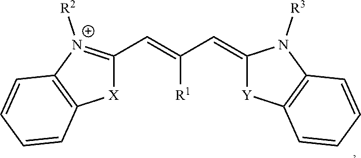
X and Y are O, R¹ is ethyl, and R² and R³ are identical and are methyl or ethyl; 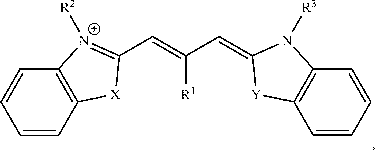
X and Y are C(CH₃)₂, R¹ is H, and R² and R³ are identical and are C₁₋₆ hydrocarbons; 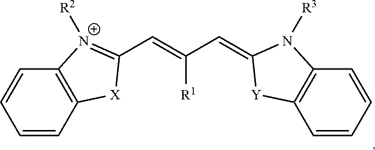
X and Y are C(CH₃)₂, R¹ is methyl, and R² and R³ are ethyl, 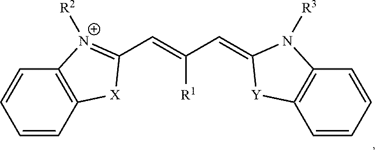
X and Y are C(CH₃)₂, R¹ is ethyl, and R² and R³ are identical and are methyl or ethyl; 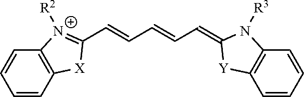
X and Y are S, and R² and R³ are identical and are C₁₋₅ hydrocarbons; 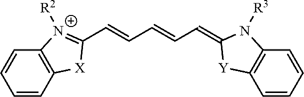
X and Y are O, and R² and R³ are identical and are ethyl, propyl, or pentyl; 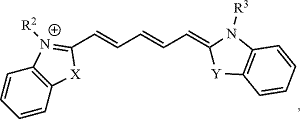
X and Y are C(CH₃)₂, and R² and R³ are identical and are methyl, ethyl, or propyl; 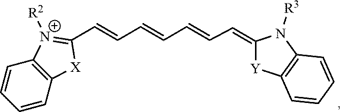
X and Y are 0, and R² and R³ are identical and are methyl or ethyl, 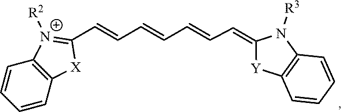
X and Y are S, and R² and R³ are identical and are C₁₋₅ hydrocarbons; or 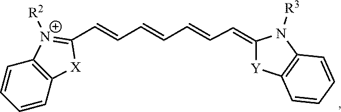
X and Y are C(CH₃)₂, and R² and R³ are identical and are methyl or ethyl.

40-41. (canceled)

42. The compound of claim 39, wherein at least one of the following applies:
(a) R¹ is H,
(b) R² and R³ are -C₁₋₅-C(=O)OR;
(c) R² and R³ are -(CH₂)₄-C(=O)OR;
(d) n = 0.

43-45. (canceled)

46. A compound of Formula Ia, a pharmaceutically acceptable salt thereof, or a geometric isomer thereof: 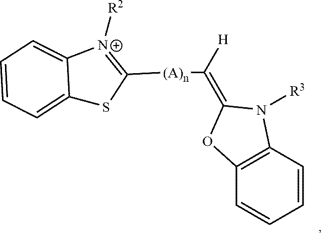
wherein:
A is 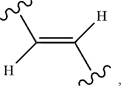
wherein one hydrogen atom in (A)ₙ is optionally substituted by R¹;
R¹ is selected from the group consisting of H, C₁₋₅ hydrocarbyl, C₁₋₅ hydrocartivl-C(=O)OR, and C₁₋₅ alkoxy;
R² and R³ are each independently selected from the group consisting of C₁₋₅ hydrocarbyl, C₁₋₅ hydrocarbyl-C(=O)OR, and C₁₋₅ alkoxy;
each occurrence of R is independently H, C₁₋₄ alkyl, or C₆₋₁₀ aryl; and
n is 0, 1, 2, or 3.

47. The compound of claim 46, wherein R² and R³ are ethyl.

48. A method for predicting cancer drug response in a subject, the method comprising:
obtaining the results of molecular data from a subject’s tissue sample, using a response prediction model on the data and applying results of the model to select a particular treatment for cancer, wherein the particular treatment comprises a treatment with a compound of Formula I or a pharmaceutically acceptable salt or geometric isomer thereof, wherein the correlation is obtained by:
i. identifying, based on gene, protein or metabolic expression levels, at least one gene product, protein, metabolite, cellular hallmark pathway, or one cellular genetic pathway involved in mitochondrial activity; and
ii. creating a regression or correlation model derived from experimental activity data of the compound of Formula I on the at least one cellular pathway or gene product in the subject’s tissue sample,
wherein the compound of Formula I has the structure: 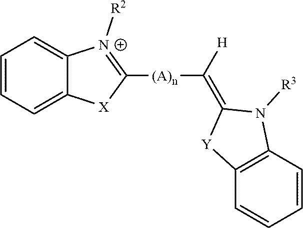
wherein:
X and Y are each independently selected from the group consisting of O, S, CH₂, and C(CH₃)₂;
A is 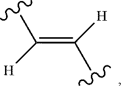
wherein one hydrogen atom in (A)ₙ is optionally substituted by R¹;
R¹ is selected from the group consisting of C₁₋₅ hydrocarbyl, C₁₋₅ hydrocarbyl-C(=O)OR, and C₁₋₅ alkoxy;
R² and R³ are each independently selected from the group consisting of C₁₋₅ hydrocarbyl, C₁₋₅ hydrocarbyl-C(=O)OR, and C₁₋₅ alkoxy; each occurrence of R is independently H, C₁₋₄ alkyl, or C₆₋₁₀ aryl; and
n is 0, 1, 2, or 3.

49. The method of claim 48, wherein at least one of the following applies:
(a) the treatment response prediction is used to treat cancer in the subject by obtaining molecular data from the subject’s tissue sample; and
treating the subject with the compound of Formula I if the response model predicts a potency of the compound of Formula I on the tissue sample above a predetermined threshold level, optionally wherein the predetermined threshold level is a predicted -log(IC₅₀) of at least 5.0;
(b) the model is based on experimental activity of at least one compound of Formula I on the a gene set containing at least one gene target or signature from Tables 1A-1C;
(c) the model is based on experimental activity of at least one compound of Formula I on a molecular expression model containing at least the MYO1B and CPT1A genes.

50-52. (canceled)

53. The method of claim 48, wherein the compound of Formula I is a halide salt of 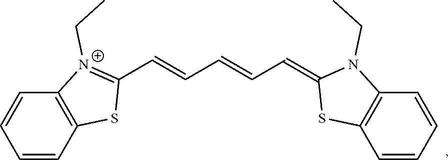
wherein the halide salt is optionally an iodide salt.

54. (canceled)

55. The method of claim 48, wherein the compound is not 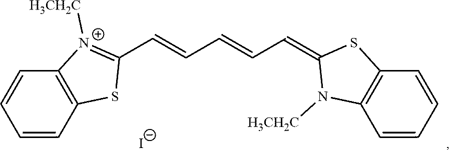
and
the cancer is selected from group consisting of liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer.

56. The method of claim 48, wherein the compound is 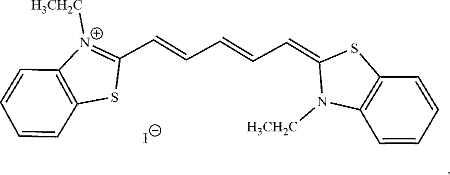
and
the cancer is selected from the group consisting of liver cancer, rectal cancer, gastric cancer, esophageal cancer, anal cancer, non-melanoma skin cancer, small intestine cancer, bile duct cancer, periampullary cancer, endometrial cancer, cervical cancer, small cell lung cancer, sarcoma, neuroendocrine cancer, lymphoma, hepatoblastoma, Wilm’s tumor and primary mesothelial cancer.

57. A method of treating, ameliorating, or preventing a cancer in a subject in need thereof, the method comprising:
administering to the subject a therapeutically effective amount of a compound of Formula II, a pharmaceutically acceptable salt thereof, or a geometric isomer thereof, optionally wherein the compound is administered to the subject as part of a composition, optionally wherein the composition is administered orally or parenterally to the subject and optionally wherein the pharmaceutically acceptable salt is a halide salt : 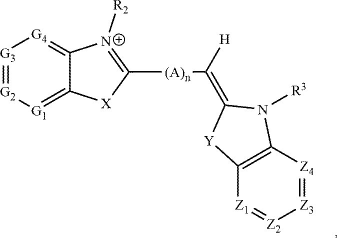
wherein:
each of G₁, G₂, G₃, and G₄ is independently C(=O), CR′, CNR″₂, N, or N-R;
each of Z₁, Z₂, Z₃, and Z₄ is independently C(=O), CR′, CNR″₂, N, or N-R″;
X and Y are each independently selected from the group consisting of O, N, S, CH₂, and C(CH₃)₂;
A is 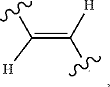
wherein one hydrogen atom in (A)ₙ is optionally substituted by R¹;
R¹ is selected from the group consisting of C₁₋₅ hydrocarbyl, C₁₋₅ hydrocarbyl-C(=O)OR, and C₁₋₅ alkoxy;
R² and R³ independently selected from the group consisting of C₁₋₅ hydrocarbyl, C₁₋₅ hydrocarbyl-C(=O)OR, and C₁₋₅ alkoxy;
each occurrence of R′ is independently H or C₁₋₄ alkyl;
each occurrence of R″ is independently H or C₁₋₄ alkyl;
each occurrence of R is independently H, C₁₋₄ alkyl, or C₆₋₁₀ aryl; and
n is 0, 1, 2, or 3.

58-59. (canceled)

60. The method of claim 57, wherein the composition comprises an encapsulating agent, optionally wherein at least one the following applies:
(a) the encapsulating agent comprises a nanoparticle or a liposome,
(b) the encapsulating agent comprises a micelle, a nucleic-acid based dendrimer nanocarrier, a protein-based nanocarrier, a polymer-based nanoparticle, a lipid-polymer based nanoparticle, a metal-based nanoparticle, a nanocrystal, a liposomal carrier, a carbon nanotube based nanoparticle, or a polymeric micelle;
(c) the encapsulating agent comprises mammalian serum albumin, which is optionally of human origin;
(d) the encapsulating agent further comprises a targeting agent, which optionally comprises
(i) a peptide ligand, a nucleotide ligand, a polysaccharide ligand, a fatty acid ligand, a lipid ligand, a small molecule ligand, an antibody, an antibody fragment, an antibody mimetic, or an antibody mimetic fragment or
(ii) folic acid or transferrin.

61-69. (canceled)

70. The method of claim 57, wherein the cancer is selected from group consisting of liver cancer, colon cancer, rectal cancer, gastric cancer, esophageal cancer, head and neck cancer, renal cancer, breast cancer, ovarian cancer, endometrial cancer, cervical cancer, anal cancer, malignant melanoma, non-melanoma skin cancer, brain cancer, prostate cancer, small intestine cancer, bile duct cancer, periampullary cancer, pancreatic cancer, lung cancer, sarcoma, thyroid cancer, neuroendocrine cancer, leukemia, lymphoma, hepatoblastoma, Wilm’s tumor, glioblastoma and primary mesothelial cancer.

71. The method of claim 57, wherein the compound is administered at an advanced stage of peritoneal involvement by a cancer selected from the group consisting of liver cancer, rectal cancer, gastric cancer, esophageal cancer, head& neck cancer, renal cancer, breast cancer, ovarian cancer, endometrial cancer, cervical cancer, anal cancer, skin cancer, prostate cancer, small intestine cancer, bile duct cancer, pancreatic cancer, lung cancer, sarcoma, neuroendocrine cancer, and primary mesothelial cancer,
optionally wherein the neuroendocrine cancer is selected from the group consisting of adrenocortical carcinoma, a carcinoid tumor, a pancreatic neuroendocrine tumor, and thymic cancer.

72. (canceled)

73. The method of claim 57, further comprising administering at least one other chemotherapeutic agent,
wherein the at least one other chemotherapeutic agent optionally comprises a DNA damaging agent, a microtubule inhibitor, a Topoisomerase inhibitor, an anti-metabolite , a cell cycle checkpoint inhibitor, a mechanistic target of rapamycin kinase (mTOR) inhibitor, a receptor tyrosine kinase (RTK) inhibitor, a DNA damage repair inhibitor, a ATM or ATR inhibitor, a Non-Homologous End Joining (NHEJ) inhibitor, a Poly(ADP) ribose glycohydrolase (PARG) inhibitor, DNA Methyl Transfrase inhibitors (DNMT), an anti-cell surface protein, an immune checkpoint inhibitor, an anti-diabetic drug, an immune modulator, or a combination chemotherapy.

74. (canceled)

75. The method of claim 74, wherein at least one of the following applies:
(a) the DNA damaging agent comprises 5-Fluorouracil, Gemcitabine, Cytarabine, Capecitabine, Dacarbazine, Temozolomide, Mitomycin C, Paclitaxel, Docetaxel, nab-Paclitaxel, Ifosfamide, Cyclophosphamide, Adriamycin, Doxorubicin, Cisplatin, Carboplatin, or Oxaliplatin;
(b) microtubule inhibitor comprises Vincristine, Vinorelbine, or Vinblastine;
(c) the topoisomerase inhibitor comprises Topotecan, Irinotecan, or Etoposide,
(d) the CDK inhibitor comprises an inhibitor of CDK4, an inhibitor of CDK6, or an inhibitor of CDK4 and CDK6;
(e) the DNA damage repair inhibitor comprises Olaparib, Veliparib, Talazoparib, Rucaparib, or Niraparib;
(f) the Cell cycle checkpoint inhibitor comprises a WEE1 inhibitor,
(g) the DNMT inhibitor comprises a Azacytadine, Decitabine, Glyburide, or Panobinostat;
(h) the anti-diabetic drug comprises Metformin, Acarbose, Miglitol, a Sodium-glucose transporter (SGLT) inhibitor, or a Thiazolidinedione;
(i) the ATR or ATM inhibitor comprises VE-821, M6620, AZD1390, or BAY-1895344.

76-83. (canceled)

84. The method of claim 57, wherein at least one of the following applies:
(a) the method further comprises administering to the subject at least one hormone antagonist, selective hormone receptor modulating agent, or hormone enzyme inhibitor_{;}
(b) the method further comprises administering radiation therapy to the subject;
(c) the method alleviates a symptom of the cancer.

85-86. (canceled)

87. A compound of Formula II, or a pharmaceutically acceptable salt thereof, or a geometric isomer thereof: 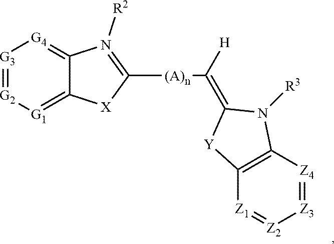
wherein:
each of G₁, G₂, G₃, and G₄ is independently C(=O), CR′, CNR″₂, N, or N-R″;
each of Z₁, Z₂, Z₃, and Z₄ is independently C(=O), CR′, CNR″₂, N, or N-R″;
X and Y are each independently selected from the group consisting of O, N, S, CH₂, and C(CH₃)₂;
A is 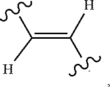
wherein one hydrogen atom in (A)ₙ is optionally substituted by R¹;
R¹ is selected from the group consisting of C₁-₅ hydrocarbyl, C₁₋₅ hydrocarbyl-C(=O)OR, and C₁₋₅ alkoxy;
R² and R³ are each independently selected from the group consisting of C₁₋₅ hydrocarbyl, C₁₋₅ hydrocarbyl-C(=O)OR, and C₁₋₅ alkoxy;
each occurrence of R′ is independently H or C₁₋₄ alkyl;
each occurrence of R″ is independently H or C₁₋₄ alkyl;
each occurrence of R is independently H, C₁₋₄ alkyl, or C₆₋₁₀ aryl; and
n is 0, 1, 2, or 3.

88. The compound of claim 87, wherein the compound is selected from the group consisting of 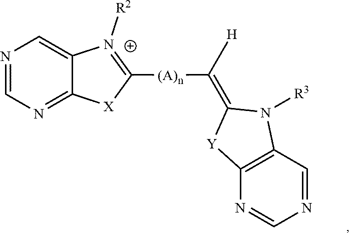 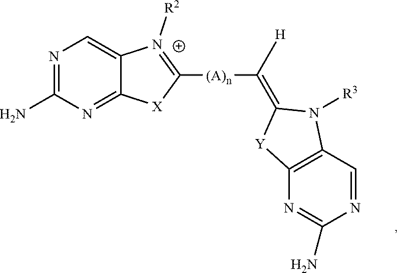
and 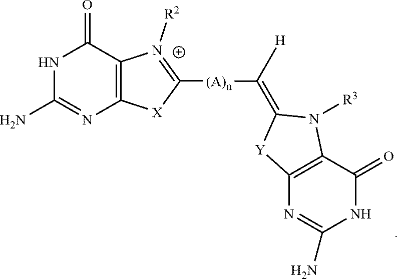
.

89. A method for predicting cancer drug response in a subject, the method comprising:
obtaining the results of molecular data from a subject’s tissue sample, using a response prediction model on the data and applying results of the model to select a particular treatment for cancer, wherein the particular treatment comprises a treatment with a compound of Formula II or a pharmaceutically acceptable salt or geometric isomer thereof, wherein the correlation is obtained by:
i. identifying, based on gene, protein or metabolic expression levels, at least one gene product, protein, metabolite, cellular hallmark pathway, or one cellular genetic pathway involved in mitochondrial activity; and
ii. creating a regression or correlation model derived from experimental activity data of the compound of Formula II on the at least one cellular pathway or gene product in the subject’s tissue sample,
wherein the compound of Formula II has the structure: 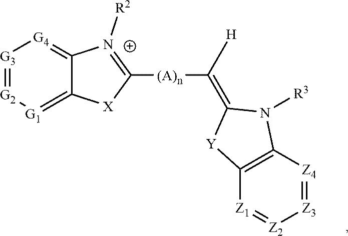
wherein:
each of G₁, G₂, G₃, and G₄ is independently C(=O), CR′, CNR″₂, N, or N-R″;
each of Z₁, Z₂, Z₃, and Z₄ is independently C(=O), CR′, CNR″₂, N, or N-R″;
X and Y are each independently selected from the group consisting of O, N, S, CH₂, and C(CH₃)₂;
A is 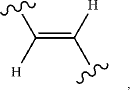
wherein one hydrogen atom in (A)ₙ is optionally substituted by R¹;
R¹ is selected from the group consisting of C₁₋₅ hydrocarbyl, C₁₋₅ hydrocarbyl-C(=O)OR, and C₁₋₅ alkoxy;
R² and R³ are each independently selected from the group consisting of C₁₋₅ hydrocarbyl, C₁₋₅ hydrocarbyl-C(=O)OR, and C₁₋₅ alkoxy;
each occurrence of R′ is independently H or C₁₋₄ alkyl,
each occurrence of R″ is independently H or C₁₋₄ alkyl;
each occurrence of R is independently H, C₁₋₄ alkyl, or C₆₋₁₀ aryl; and
n is 0, 1, 2, or 3 .

90. The method of claim 89, wherein at least one of the following applies:
(a) the treatment response prediction is used to treat cancer in the subject by obtaining molecular data from the subject’s tissue sample; and
treating the subject with the compound of Formula II if the response model predicts a potency of the compound of Formula II on the tissue sample above a predetermined threshold level, optionally wherein the predetermined threshold level is a predicted -log(IC₅₀) of at least 5.0;
(b) the model is based on experimental activity of at least one compound of Formula I on the a gene set containing at least one gene target or signature from Tables 1A-1C;
(c) the model is based on experimental activity of at least one compound of Formula I on a molecular expression model containing at least the MYO1B and CPT1A genes.

91-93. (canceled)
